(19) United States
(12) Patent Application Publication
(10) Pub. No.: US 2025/0387164 A1
(43) Pub. Date: Dec. 25, 2025

(54) Methods And Systems For Zone And Implant Planning For A Surgical Procedure

(71) Applicant: Stryker Corporation, Portage, MI (US)

(72) Inventors: Zachary S. Bolthouse, Kalamazoo, MI (US); James G. Walen, Portage, MI (US); Zygmunt Bispham Jones, Manchester (GB)

(73) Assignee: Stryker Corporation, Portage, MI (US)

(21) Appl. No.: 18/877,780

(22) PCT Filed: Jul. 3, 2023
(86) PCT No.: PCT/US2023/026831

§ 371 Date: Dec. 20, 2024

Related U.S. Application Data
(60) Provisional application No. 63/367,550, filed on Jul. 1, 2022.

(51) Int. Cl. A61B 34/10; A61B 17/00; A61B 17/17; A61B 34/20; G06T 7/11; G06T 7/246; G06T 7/73; G06T 17/20; G06T 19/00

(52) U.S. Cl. CPC A61B 34/10; A61B 34/20; G06T 7/11; G06T 7/251; G06T 7/75; G06T 17/20; G06T 19/003; A61B 2017/00119; A61B 2017/00199; A61B 2017/00402; A61B 17/1703; A61B 2034/102; A61B 2034/105; A61B 2034/107; A61B 2034/2065; G06T 2200/04; G06T 2207/20092; G06T 2207/30012; G06T 2210/41

(57) ABSTRACT

Systems and methods for mapping zones for monitoring a position of a surgical instrument during a procedure from a model vertebra to a 3D image of a patient vertebra. A model vertebra in a first coordinate system is received, the model vertebra including a plurality of model features localized in the first coordinate system and a pose of a model zone in the first coordinate system. A 3D image of a first and second vertebra of a patient in a second coordinate system is also received. The model vertebra including the model zone is mapped to the first vertebra such that a zone for the first vertebra is generated. Input indicating a revised pose of the zone for the first vertebra is received, and a zone for the second vertebra is generated based on the revised pose of the zone for the first vertebra.

### CROSS-REFERENCE TO RELATED APPLICATIONS

This application claims the benefit of and priority to U.S. Provisional Patent Application No. 63/367,550, filed Jul. 1, 2022, which is incorporated herein by reference in its entirety.

### BACKGROUND

In modern surgery, one of the most important instruments available to medical personnel are powered surgical instruments, such as cordless drills, saws, wire drivers, high speed drills, ultrasonic handpieces, or the like. Often these surgical instruments include a motor and/or processor within a handpiece or housing. The surgical instrument may include an attachment feature configured to receive a cutting attachment designed for application to a surgical site to perform a specific medical procedure. For example, a surgical drill may utilize a cutting attachment such as a drill bit, bur, or reamer for cutting bores into tissue or for selectively removing tissue such as bone. The ability to use powered surgical instruments on a patient lessens the physical strain of medical professionals when performing medical procedures. Moreover, most surgical procedures can be performed more quickly and more accurately with powered surgical instruments than with the manual equivalents that preceded them.

A surgical navigation system may assist a medical professional in navigation of surgical instruments during surgery. One or more 2D or 3D images of a spine of a patient may be acquired prior to or during a spinal surgery procedure and accessed by the surgical navigation system. Alert zone planning for a spinal surgery procedure involves establishing zones relative to the 2D or 3D images to define areas surrounding critical anatomical structures of the spine, such as a vertebra or the spinal cord, to avoid during the spinal surgery. The alert zones may be used to control operation of the surgical instruments to avoid accidentally impinging a critical anatomical structure. Existing surgical navigation systems may automatically generate the alert zones, and thereafter allow a medical professional to provide input to manually edit the one or more automatically generated alert zones. However, in the case the surgical procedure involves multiple vertebra (as is often the case), the medical professional typically has to edit the alert zones for each vertebra in which surgery is to be performed on. As a result, the alert zone planning of existing surgical navigation systems may be quite cumbersome.

Similarly, although existing surgical navigation systems may suggest an initial implant pose, a medical professional typically has to adjust the implant pose from the initial pose for each vertebra on which surgery is to be performed, and also manually edit alert zones and implants in a similar fashion between like patients according to the medical professional's own medical preferences and medical beliefs. Thus, there exists a need for optimizing alert zone and implant planning for a medical professional.

The background description provided here is for the purpose of generally presenting the context of the disclosure. Work of the presently named inventors, to the extent it is described in this background section, as well as aspects of the description that may not otherwise qualify as prior art at the time of filing, are neither expressly nor impliedly admitted as prior art against the present disclosure.

### SUMMARY

A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions.

One general aspect includes a method for mapping zones of an anatomic model, the zones for monitoring a position of a surgical instrument relative to a patient anatomic structure corresponding to the anatomic model during a surgical procedure, to a three-dimensional image of an anatomic structure of a patient is described. The method includes receiving a three-dimensional anatomic model in a first coordinate system, the three-dimensional model including a plurality of model features localized in the first coordinate system and a pose of a model zone in the first coordinate system. The method also includes receiving a three-dimensional image of an anatomic structure of a patient that corresponds to the anatomic model. The method also includes mapping the three-dimensional anatomic model including the model zone to the patient anatomic structure based on the plurality of model features localized in the first coordinate system such that a zone for the patient anatomic structure is generated in the second coordinate system. The method also includes receiving input from a medical professional, the input indicating a revised pose of the zone for the patient anatomic structure in the second coordinate system. The method also includes generating a zone for another anatomic structure of the patient, which may similarly correspond to the anatomic model and/or also be illustrated in the three-dimensional image, based on the revised pose of the zone for the patient anatomic structure, and/or adjusting the pose of the model zone based on the revised pose of the zone for the patient anatomic structure, such as for mapping the adjusted zone model to further three-dimensional images of anatomic structures of the patient corresponding to the anatomic model and/or to three-dimensional images of anatomic structures of other patients corresponding to the anatomic model. In some implementations, the anatomic model may be a vertebra model, and the patient anatomic structure may be a vertebra of a patient. Other examples of this aspect include corresponding computer systems, apparatus, and computer programs recorded on one or more computer storage devices, each configured to perform the actions of the methods.

One general aspect includes a method for mapping zones to a three-dimensional medical image for controlling a surgical instrument is described. The method includes retrieving a three-dimensional vertebra model in a first coordinate system, the three-dimensional vertebra model including (i) a plurality of model features localized in the first coordinate system and (ii) a pose of a model zone relative to a critical structure for the surgical instrument in the first coordinate system. The method also includes retrieving a three-dimensional image in a second coordinate system, the three-dimensional image representing a first vertebra and a second vertebra. The method also includes mapping the three-dimensional vertebra model including the model zone to the first vertebra based on the plurality of model features localized in the first coordinate system such that a zone for the first vertebra is generated. The method also includes receiving input from a medical professional, the input indicating a revised pose of the zone of the first vertebra in the second coordinate system. The method also includes generating a zone for the second vertebra based on the revised pose of the zone of the first vertebra. Other examples of this aspect include corresponding computer systems, apparatus, and computer programs recorded on one or more computer storage devices, each configured to perform the actions of the methods.

One general aspect includes a method for mapping zones for a surgical instrument to a three-dimensional medical image is described. The method includes retrieving a three-dimensional vertebra model in a first coordinate system, the three-dimensional vertebra model including a plurality of model features localized in the first coordinate system and a pose of a model zone for the surgical instrument in the first coordinate system. The method also includes retrieving a three-dimensional image having a first vertebra and a second vertebra in a second coordinate system. The method also includes mapping the three-dimensional vertebra model including the model zone to the first vertebra in order to generate a zone for the first vertebra and a zone for the second vertebra. The method also includes receiving input from a medical professional, the input indicating a revised pose for the model zone of the three-dimensional vertebra model in the first coordinate system. The method also includes propagating the revised pose for the model zone to at least one of the zone for the first vertebra and the zone for the second vertebra. Other examples of this aspect include corresponding computer systems, apparatus, and computer programs recorded on one or more computer storage devices, each configured to perform the actions of the methods.

One general aspect includes a method for mapping zones for a surgical instrument to a three-dimensional image, is described. The method includes retrieving a three-dimensional vertebra model in a first coordinate system, the three-dimensional vertebra model including (i) a plurality of model features localized in the first coordinate system and (ii) a pose of a model zone for the surgical instrument in the first coordinate system. The method also includes retrieving a three-dimensional image of a first patient having at least one vertebra in a second coordinate system. The method also includes mapping the three-dimensional vertebra model including the model zone to the at least one vertebra to generate a zone for the at least one vertebra. The method also includes receiving input with respect to a revised pose of the zone for the at least one vertebra. The method also includes revising the pose of the model zone based on the revised pose of the zone for the at least one vertebra. The method also includes retrieving a three-dimensional image of a second patient having at least one vertebra in a third coordinate system. The method also includes mapping the three-dimensional vertebra model including the revised pose of the model zone to the at least one vertebra of the second patient in order to generate a zone for the at least one vertebra of the second patient. Other examples of this aspect include corresponding computer systems, apparatus, and computer programs recorded on one or more computer storage devices, each configured to perform the actions of the methods.

One general aspect includes a method for adjusting a zone for a three-dimensional image according to historical preference of a medical professional is described. The method also includes retrieving a three-dimensional image having at least one vertebra in a first coordinate system. The method also includes receiving a three-dimensional vertebra model in a second coordinate system, the three-dimensional vertebra model including a pose of a model zone for a surgical instrument in the second coordinate system and a plurality of model features localized in the second coordinate system. The method also includes mapping the three-dimensional vertebra model including the model zone to the at least one vertebra based on the plurality of model features localized in the second coordinate system such that a zone for the at least one vertebra is generated. The method also includes receiving input from the medical professional with respect to a revised pose of the zone for the at least one vertebra. The method also includes determining one or more transformations based on the pose of the model zone of the three-dimensional vertebra model and the revised pose of the zone for the at least one vertebra in the first coordinate system. The method also includes storing the one or more transformations based on the pose of the model zone for the three-dimensional vertebra model in the first coordinate system and the revised pose of the zone for the at least one vertebra in the first coordinate system in a database including transformation data for a plurality of patients. The method also includes determining correction data based on the transformation data for the plurality of patients. The method also includes retrieving a three-dimensional image having at least one vertebra of a second patient in a third coordinate system. The method also includes mapping the three-dimensional vertebra model including the model zone to the at least one vertebra of the second patient based on the plurality of model features localized in the third coordinate system and based on the correction data such that a zone for the at least one vertebra of the second patient is generated. Other examples of this aspect include corresponding computer systems, apparatus, and computer programs recorded on one or more computer storage devices, each configured to perform the actions of the methods.

One general aspect includes a method for adjusting a zone for a three-dimensional image according to historical preference of a medical professional is described. The method includes receiving a three-dimensional image having at least one vertebra in a first coordinate system. The method also includes receiving a three-dimensional vertebra model in a second coordinate system, the three-dimensional vertebra model including a pose of a model zone relative for a surgical instrument in the second coordinate system and a plurality of model features localized in the second coordinate system. The method also includes mapping the three-dimensional vertebra model including the model zone to the at least one vertebra based on the plurality of model features localized in the second coordinate system such that a zone for the at least one vertebra is generated. The method also includes receiving input from the medical professional with respect to a revised pose of the zone for the at least one vertebra. The method also includes determining one or more transformations based on the pose of the model zone of the three-dimensional vertebra model and the revised pose of the zone for the at least one vertebra in the first coordinate system. The method also includes storing the one or more transformations based on the pose of the model zone for the three-dimensional vertebra model in the first coordinate system and the revised pose of the zone for the at least one vertebra in the first coordinate system in a database including transformation data for a plurality of patients. The method also includes determining correction data based on the transformation data for the plurality of patients. The method also includes selectively adjusting the pose of the model zone of the three-dimensional vertebra model based on the correction data. Other examples of this aspect include corresponding computer systems, apparatus, and computer programs recorded on one or more computer storage devices, each configured to perform the actions of the methods.

One general aspect includes a method for adjusting a zone for a three-dimensional image according to historical preference of a medical professional is described. The method includes retrieving a three-dimensional image having at least one vertebra in a first coordinate system. The method also includes retrieving a three-dimensional vertebra model in a second coordinate system, the three-dimensional vertebra model including a pose of a model zone for a surgical instrument in the second coordinate system. The method also includes mapping an initial pose of a zone for the at least one vertebra of the three-dimensional image based on the pose of the model zone of the three-dimensional vertebra model. The method also includes receiving input from the medical professional with respect to a revised pose of the zone for the at least one vertebra. The method also includes comparing the revised pose of the zone for the at least one vertebra relative the initial pose of the zone for the at least one vertebra. The method also includes storing the comparison of the revised pose of the zone for the at least one vertebra relative to the initial pose for the at least one vertebra in a zone correction database. The method also includes learning a zone preference for the medical professional based on the zone correction database. The method also includes adjusting a pose of a zone for at least one vertebra of a second patient based on the learned zone preference. Other examples of this aspect include corresponding computer systems, apparatus, and computer programs recorded on one or more computer storage devices, each configured to perform the actions of the methods.

One general aspect includes a method for adjusting a planned implant based on historical preference of a medical professional is described. The method includes retrieving a three-dimensional image of a first patient, the three-dimensional image including at least one vertebra in a first coordinate system. The method also includes retrieving a three-dimensional vertebra model in a second coordinate system, the three-dimensional vertebra model including a pose of a model implant in the second coordinate system. The method also includes mapping the three-dimensional vertebra model including the pose of the model implant to the at least one vertebra to generate an initial pose of the planned implant for the at least one vertebra of the three-dimensional image. The method also includes receiving input from the medical professional indicating a correction for the planned implant, the correction including a revised pose for the planned implant. The method also includes determining one or more transformations based on the initial pose of the planned implant and the revised pose of the planned implant. The method also includes storing the one or more transformations based on the initial pose of the planned implant and the revised pose of the planned implant for the at least one vertebra in a database including implant transformation data for a number of patients. The method also includes determining correction data based on the implant transformation data for the number of patients. The method also includes selectively adjusting the pose of the model implant for the three-dimensional vertebra model based on the correction data. Other examples of this aspect include corresponding computer systems, apparatus, and computer programs recorded on one or more computer storage devices, each configured to perform the actions of the methods.

One general aspect includes a method for adjusting a pose of a planned implant based on historical preference of a medical professional is described. The method includes retrieving a three-dimensional image of a first patient, the three-dimensional image including at least one vertebra in a first coordinate system. The method also includes retrieving a three-dimensional vertebra model in a second coordinate system, the three-dimensional vertebra model including a pose of a model implant in the second coordinate system. The method also includes mapping the three-dimensional vertebra model including the pose of the model implant to the at least one vertebra to generate an initial pose of the planned implant. The method also includes receiving input from the medical professional indicating a correction of a revised pose of the planned implant. The method also includes determining one or more transformations based on the initial pose of the planned implant and the revised pose of the planned implant. The method also includes storing the one or more transformations based on the initial pose and the revised pose of the planned implant in a database including implant transformation data for a plurality of patients. The method also includes determining correction data based on the implant transformation data for the plurality of patients. The method also includes storing the correction data in an implant correction database. The method also includes retrieving a three-dimensional image of a second patient, the three-dimensional image including at least one vertebra in a third coordinate system. The method also includes mapping the three-dimensional vertebra model including the pose of the model implant to the at least one vertebra of the three-dimensional image of the second patient to generate a pose of the planned implant for the at least one vertebra of the three-dimensional image of the second patient. The method also includes selectively adjusting the pose of the planned implant based on the correction data. Other examples of this aspect include corresponding computer systems, apparatus, and computer programs recorded on one or more computer storage devices, each configured to perform the actions of the methods.

One general aspect includes a method for adjusting a zone based on historical preference of a medical professional is described. The method includes retrieving a three-dimensional image of a first patient, the three-dimensional image including at least one vertebra in a first coordinate system. The method also includes retrieving a three-dimensional vertebra model in a second coordinate system, the three-dimensional vertebra model including a plurality of poses for a plurality of model zones in the second coordinate system. The method also includes retrieving one or more preferences associated with a previous procedure conducted by the medical professional. The method also includes mapping the three-dimensional vertebra model including at least one pose of the plurality of model zones to the at least one vertebra based on the one or more preferences associated with the previous procedure so that at least one zone for the at least one vertebra is generated. Other examples of this aspect include corresponding computer systems, apparatus, and computer programs recorded on one or more computer storage devices, each configured to perform the actions of the methods.

Further areas of applicability of the present disclosure will become apparent from the detailed description, the claims, and the drawings. The detailed description and specific examples are intended for purposes of illustration only and are not intended to limit the scope of the disclosure.

### BRIEF DESCRIPTION OF THE DRAWINGS

The present disclosure will become more fully understood from the detailed description and the accompanying drawings.

FIG. 1 illustrates an exemplary layout of an operating room including at least one surgical instrument assembly and a surgical navigation system for performing a medical procedure on a patient, according to the teachings of the present disclosure.

FIG. 2 illustrates a surgical system including a plurality of surgical instrument assemblies and a surgical navigation system for tracking a surgical instrument associated with each of the various surgical instrument assemblies, according to the teachings of the present disclosure.

FIG. 3 illustrates a hand-held surgical instrument, the hand-held surgical instrument comprising a battery and a plurality of end effectors, according to the teachings of the present disclosure.

FIG. 4 illustrates an exemplary graphical user interface of a navigation system, the graphical user interface displaying a lumbar vertebra region selected by the medical professional to be segmented with the navigation system, according to the teachings of the present disclosure.

FIG. 5 illustrates an exemplary graphical user interface of a navigation system, the graphical user interface displaying a vertebra model in various views, one view including a plurality of model alert zones and a plurality of model virtual boundaries, another view including a first model implant and a second model implant, and a third view including a plain view of the vertebra model without implants, alert zones, or boundaries, according to the teachings of the present disclosure.

FIG. 6 illustrates an exemplary graphical user interface of a navigation system, the graphical user interface displaying a vertebra of a segmented lumbar vertebra region subject to be operated on with the surgical system, according to the teachings of the present disclosure.

FIG. 7 illustrates an exemplary graphical user interface of a navigation system, the graphical user interface including user-selectable objects related to planning and/or execution of a surgical procedure, according to the teachings of the present disclosure.

FIG. 8 illustrates an exemplary graphical user interface of a navigation system, the graphical user interface displaying suggested poses, in various views, for a first implant and a second implant of a vertebra of a segmented lumbar vertebra region subject to be operated on with the surgical system, according to the teachings of the present disclosure.

FIG. 9 illustrates an exemplary graphical user interface of a navigation system, the graphical user interface displaying a revised pose, in various views, for a first implant of a vertebra of a segmented lumbar vertebra region subject to be operated on with the surgical system, according to the teachings of the present disclosure.

FIG. 10 illustrates an exemplary graphical user interface of a navigation system, the graphical user interface displaying a suggested pose, in various views, for a first alert zone of a vertebra of a segmented lumbar vertebra region subject to be operated on with the surgical system, according to the teachings of the present disclosure.

FIG. 11 illustrates an exemplary graphical user interface of a navigation system, the graphical user interface displaying a revised pose, in various views, for a first alert zone of a vertebra of a segmented lumbar vertebra region subject to be operated on with the surgical system, according to the teachings of the present disclosure.

FIG. 12 illustrates an exemplary method performed by the navigation system to generate an alert zone for a vertebra according to the teachings of the present disclosure.

FIG. 13 illustrates another exemplary method performed by the navigation system to generate an alert zone for a vertebra according to the teachings of the present disclosure.

FIGS. 14A and 14B illustrate another exemplary method performed by the navigation system to generate an alert zone for a vertebra according to the teachings of the present disclosure.

FIGS. 15A and 15B illustrate an exemplary method performed by the navigation system to adjust an alert zone for a model vertebra according to the teachings of the present disclosure.

FIG. 16 illustrates an exemplary method performed by the navigation system to adjust an alert zone for a vertebra according to the teachings of the present disclosure.

FIGS. 17A and 17B illustrate an exemplary method performed by the navigation system to adjust a pose of an implant for a model vertebra according to the teachings of the present disclosure.

FIGS. 18A and 18B illustrate an exemplary method performed by the navigation system to adjust a pose of an implant for a vertebra according to the teachings of the present disclosure.

FIG. 19 illustrates another exemplary method performed by the navigation system to adjust an alert zone for a vertebra according to the teachings of the present disclosure.

In the drawings, reference numbers may be reused to identify similar and/or identical elements.

### DETAILED DESCRIPTION

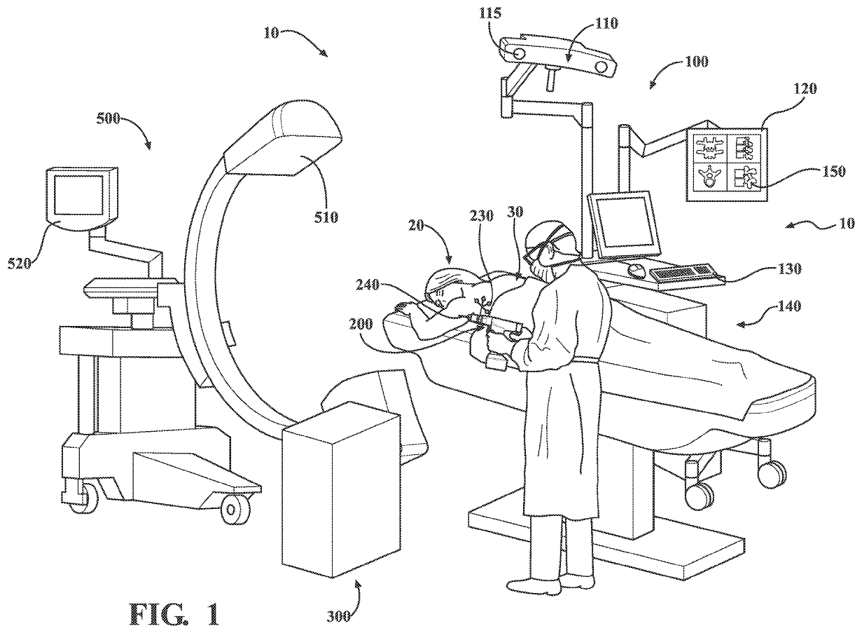

With reference to FIG. 1, an exemplary configuration of an operating room or surgical suite for performing a medical procedure on a patient 20 using the surgical system 10 is shown. The surgical navigation system 100 may include a navigation computer 140, user input devices 130, a display unit 120, and a tracking unit 110. The navigation computer 140 may include a central processing unit (CPU) and/or other processors, memory (not shown), and storage (not shown). The navigation computer 140 may be a personal computer, laptop computer, tablet computer or any other suitable computing device. The navigation computer 140 may include surgical navigation software including one or more modules and/or operating instructions related to the operation of the surgical navigation system 100 and to implement the various routines, functions, or methods disclosed herein.

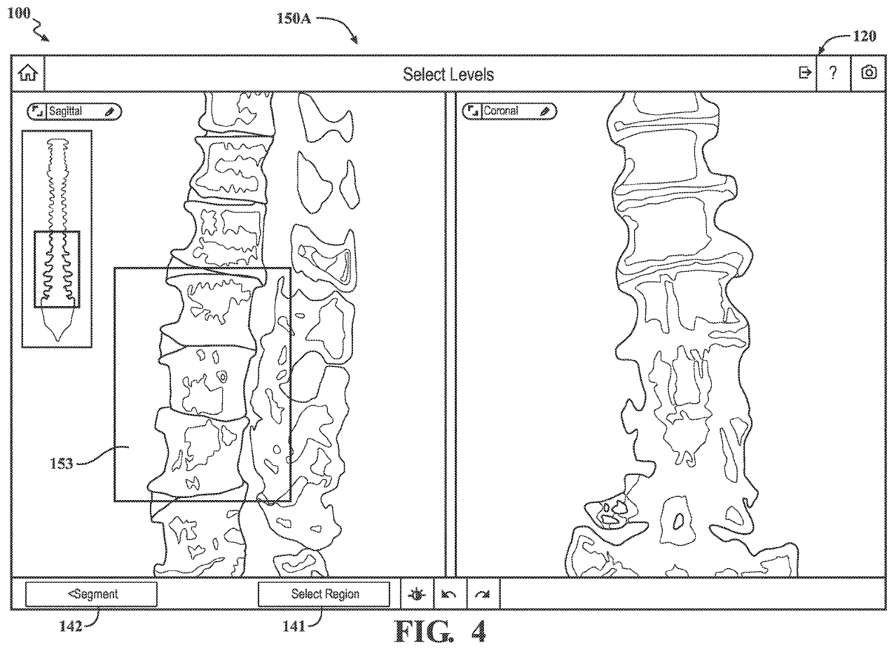

The display unit 120 is configured to display various graphical user interfaces (GUI) 150 and patient images (e.g., pre-operative patient images or intraoperative patient images). The pre-operative images may be uploaded to the surgical navigation system 100 prior to the surgical procedure. A medical professional may interact with the various GUIs 150 via user input devices 130 or via touch input. In particular, the various GUIs 150 will be discussed in greater detail with respect to FIGS. 4-11. The display unit 120 of the surgical navigation system 100 may be configured to display various prompts or data entry boxes. For example, the display unit 120 may be configured to display a text box or prompt that allows the medical professional to manually enter or select the type of surgical procedure to be performed.

The display unit 120 may be further configured to display a surgical plan for a medical procedure overlaid on the patient images. The surgical plan may include the surgical pathway for executing the medical procedure or planned trajectory or orientation for the medical instrument during the medical procedure. The surgical plan may also include a pose of an implant or medical device to be inserted during the medical procedure overlaid onto the patient data or image. It is contemplated that the surgical navigation system 100 may be configured to display and/or project a holographic image of surgical pathway for executing the medical procedure or planned trajectory or orientation for the medical instrument during the medical procedure. This may include projecting the surgical pathway onto the patient 20 or other surface in the operating room. It may also include a projection of the surgical pathway onto the head unit worn by the medical professional, such as a lens, shield, or glasses of the head unit. An exemplary configuration of the surgical navigation system 100 including a display unit worn by the medical professional to display the target trajectory and/or target location is disclosed in International Publication No. WO/2018/203304 A1, the entirety of which is hereby incorporated by reference.

The GUI 150 may be configured to allow the medical professional to input or enter patient data or modify the surgical plan. The patient data, in addition to the patient images, may include additional information related to the type of medical procedure being performed, the patient's anatomical features, the patient's specific medical condition, and/or operating settings for the surgical navigation settings. For example, in performing a spinal fusion procedure, the medical professional may enter information via the user input devices 130 and/or the GUI 150 related to the specific vertebra or vertebra on which the medical procedure is being performed. The medical professional may also input various anatomical dimensions related to the vertebrae and/or the size and shape of a medical device or implant to be inserted during the medical procedure. The user input devices 130 and/or the GUI 150 may also be configured to allow the medical professional to select, edit or manipulate the patient data. For example, the medical professional may identify and/or select anatomical features from the patient data. This may include selecting the surgical site, such as selecting the vertebra and/or specific area on the vertebra where the medical procedure is to be performed.

The surgical navigation system 100 may be configured to utilize segmentation to facilitate the generation of alert zones of interests around critical anatomical features. These critical anatomical features may include, cortical walls, nerves, blood vessels or similar critical anatomical structures. The alert zones may be defined by one or more virtual boundaries. The medical professional may also provide input to the user input devices 130 or to the GUI 150 to identify additional critical anatomical features and/or alert zones in addition to what was suggested by the navigation computer 140 or wish to edit alert zones and/or virtual boundaries generated by the navigation computer 140. The medical professional may also provide input to the user input devices 130 or to the GUI 150 to select and/or input a target location, target trajectory, target depth or similar feature of the surgical pathway to help guide the medical professional in performing the medical procedure.

The input to the user input devices 130 or to the GUI 150 may be provided to select the surgical instrument to be used, to select the device and/or implant to be inserted, to select a planned pose where the device or implant is to be placed within the patient, and to allow the medical professional to select the parameters of the implant to be inserted, such as the length and/or diameter of the screw to be inserted, as will be discussed in greater detail below.

The surgical system 10 may also include an imaging system 500 and a surgical navigation system 100. The imaging system 500, such as CT or MRI imaging device, may perform intraoperative imaging. The imaging system 500 may include a scanner 510 and a display unit 520. The scanner 510 may be utilized to take an image of the surgical site 30 on the patient 20 and display it on the display unit 520. For example, the scanner 510 may include a C-arm configured to be rotated about the patient 20 to produce a plurality of images of the surgical site 30. The imaging system 500 may also include a processor (not shown) including software, as is known by those skilled in the art, which is capable of taking the plurality of images captured by the scanner 510 and producing a 2D image and/or a 3D model of the surgical site 30. The display unit 520 may be configured to display the resulting 2D image and/or 3D model.

The imaging system 500 may also be in communication with the navigation computer 140 of the surgical navigation system 100. The imaging system 500 may be configured to communicate via a wired and/or a wireless connection with the navigation computer 140. For example, the imaging system 500 may be configured to provide pre-operative and/or intra-operative image data, such as the resulting 2D image and/or 3D model of the surgical site 30, to the navigation computer 140 to provide the resulting 2D image and/or 3D model to the display unit 120.

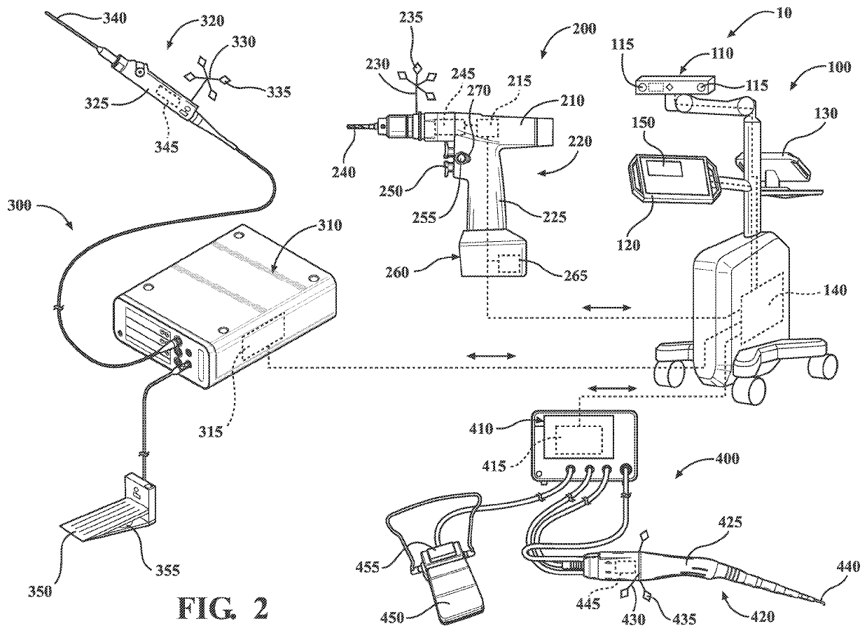

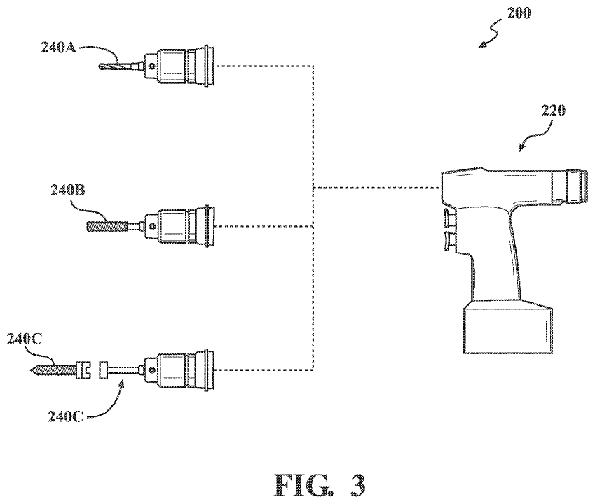

The surgical system 10 also includes at least one of the surgical instrument assembly 200 in wired or wireless communication with the navigation computer 140 directly, or indirectly. While only the first surgical instrument assembly 200 is illustrated in FIG. 1, it should be understood that it is only an exemplary configuration of the surgical system 10, and that it is contemplated that any number of surgical instrument assemblies 200, 300, 400 (as described in further detail with respect to FIG. 2) may be positioned within the operating room. The first surgical instrument assembly 200 includes the first surgical instrument 220 including the end-effector 240 and the tracking device 230. The tracking device 230 includes a plurality of markers 235 that are capable of being identified and/or tracked by the surgical navigation system 100. Reliable tracking of surgical instruments during the execution of surgical procedures to follow the planned surgical pathway and/or to avoid critical anatomical structures is of the utmost importance. Furthermore, providing feedback and/or notifying the medical professional executing the procedure when the surgical instrument becomes misaligned with the surgical pathway and/or is at risk of impinging on a critical anatomical structure is of similar importance. With additional reference to FIG. 3, the surgical instrument 220 may be coupled to a drill chuck 240A, a tap 240B for creating threads on the interior surface of a hole or aperture, or a driver 240C for driving or inserting a screw within the borehole or aperture of the bone.

The tracking unit 110 may include one or more sensors 115 for tracking the tracking device 230 of the surgical instrument assembly 200. The sensors may include cameras, such as CCD cameras, CMOS cameras, and/or optical image cameras, magnetic sensors, radio frequency sensors, or any other sensor adapted to detect and/or sense the position of a tracking device 230 of the surgical instrument assemblies 200. Description of a suitable tracking unit, and the various localizers that it can utilize may be found in U.S. Patent Publication No. 2017/0333137, which is hereby incorporated by reference in its entirety.

Referring to FIG. 2, various other surgical instrument assemblies 300, 400 in addition to the surgical instrument assembly 200 are illustrated in communication with the surgical navigation system 100. Each of the various exemplary surgical instrument assemblies 200, 300, 400 will be described in greater detail below. The surgical instruments assemblies 200, 300, 400 may be configured to be in wired and/or wireless communication with the surgical navigation system 100. Furthermore, each of the surgical instrument assemblies 200, 300, 400 may have a number of similar components capable of performing similar functions and/or operations. Similar components between each of the various surgical instrument assemblies 200, 300, 400 will include the same two-digit number with a leading 2, 3, or 4 to reflect the associated surgical instrument assembly 200, 300, 400. For example, each of the surgical instrument assemblies 200, 300, 400 may include a surgical instrument 220.

The first surgical instrument assembly 200 is in communication with the surgical navigation system 100. As previously discussed, the first surgical instrument assembly 200 may be configured as a first surgical instrument 220, such as a surgical drill or driver, including a handpiece 225. The handpiece 225 may include a housing 210 configured to house the components of the first surgical instrument 220. The handpiece 225 may be shaped to define a handle or grip portion for the medical professional to hold while performing a medical procedure. Suitable handpieces are described in U.S. Pat. No. 5,747,953, which is hereby incorporated by reference in its entirety.

The first surgical instrument 220 may further include a first instrument processor 215 and a motor 245. Each of the first instrument processor 215 and the motor 245 may be disposed within the handpiece 225 of the first surgical instrument 220. The first instrument processor 215 and the motor 245 may be in communication with one another, and the first instrument processor 215 may be configured to control the operation of the motor 245, and by extension the first surgical instrument 220. For example, the first surgical instrument 220 may include an end-effector 240, such as a drill bit for boring a hole or a driver for inserting a screw. The end-effector 240 may be coupled to the handpiece 225 of the first surgical instrument 220 such that the motor 245 may be operably coupled to the end-effector 240. For example, the motor 245 may be configured to rotate a drill bit 240 to bore a hole and/or remove biological tissue. The first instrument processor 215 may be in communication with the motor 245 and configured to control operation of the motor 245, and by extension the drill bit 240. The first instrument processor 215 may also be in communication with the navigation computer 140 and configured to exchange data related to the position and/or orientation of the first surgical instrument 220, as well as data related to the operation of the first surgical instrument 220. For example, the first instrument processor 215 and the navigation computer 140 may be configured to communicate data between one another related to the operation of the first surgical instrument 220 based on the position and/or orientation of the first surgical instrument 220 as detected by the surgical navigation system 100.

The first surgical instrument assembly 200 may also include a power source 260. The power source 260 may be removably coupled to the handpiece 225 of the surgical drill 220. For example, the power source 260 may include a removable battery pack. It is also contemplated that the power source 260 may be formed as part of, or disposed within, the handpiece 225 of the first surgical instrument 220. The power source 260 may be in electrical communication with the first instrument processor 215 and/or the motor 245 and configured to selectively provide power to the motor 245 to rotate the end-effector 240. The power source 260 may also be a surgical console providing power to the first surgical instrument 220 with a cord.

In instances where the power source 260 takes the form of a removable battery pack, the power source 260 may further include a processor 265. The processor 265 may be in communication within the first instrument processor 215 via power signals and/or data signals. The processor 265 and the first instrument processor 215 may be configured to communicate between one another to control operation of the motor 245, and by extension the first surgical instrument 220. For example, the processor 265 in the power source 260 may be configured to identify when the power source 260 has dropped below a threshold charge level such that the power source 260 may be unable to continue operating the motor 245 at a minimum threshold for boring a hole or cutting biological tissue. The processor 265 may be configured to cut off all power to the first instrument processor 215 and/or the motor 245 to prevent operation of the end-effector 240 until the power source 260 has a sufficient charge level to operate the motor 245 at a rate above the minimum threshold for boring a hole or cutting biological tissue. The processor 265 in the power source 260 may also be in wireless communication with the navigation computer 140. The power source 260 may include a transceiver configured to send and receive signals between the power source 260 and the surgical navigation system 100 and/or the instrument processor 215.

The processor 265 and the navigation computer 140 may be configured to communicate data between one another related to the operation of the first surgical instrument 220 based on the position and/or orientation of the first surgical instrument 220 as detected by the surgical navigation system 100. For example, the surgical navigation system 100 may be configured to communicate data to the processor 265 including instructions for the processor 265 to discontinue providing energy to the first instrument processor 215 and/or the motor 245 based on the position and/or orientation of the first surgical instrument 220 as detected by the surgical navigation system 100. The surgical navigation system 100 may also be configured to communicate data to the processor 265 including instructions for the processor 265 to continue and/or resume providing energy to the first instrument processor 215 and/or the motor 245 based on the position and/or orientation of the first surgical instrument 220 as detected by the surgical navigation system 100.

The first surgical instrument assembly 200 may also include a switch 250, such as a trigger or button or lever, that is operably coupled to the first instrument processor 215. The switch 250 may be configured to be manipulatable by the medical professional to control energization of the variable speed motor 245. For example, the switch 250 may be manipulatable between a first position, a deenergized state, and a second position, an energized state. The first surgical instrument assembly 200 may also include a switch sensor that is configured to detect the position of the switch 250 and produce and/or communicate a signal indicative of the position of the switch 250 to the first instrument processor 215 based on a user's manipulation of the switch 250 to control the operation of the first surgical instrument 220. For example, the switch 250 may include a first position, a second position and a plurality of intermediary positions between the first and second positions.

The first position may be configured as an off position, such that when the first instrument processor 215 receives a signal that the switch sensor has detected that the switch 250 is in the first position, the first instrument processor 215 prevents the flow of energy from the power source 260 to the motor 245, preventing the operation of the first surgical instrument 220. Alternatively, when the first instrument processor 215 receives a signal that the switch sensor has detected that the switch 250 is in the second position, the first instrument processor 215 may be configured to allow maximum flow of energy from the power source 260 to the motor 245, allowing the first surgical instrument 220 to operate at a maximum drilling or cutting speed.

When the first instrument processor 215 receives a signal that the switch sensor has detected that the switch 250 is in one of the intermediary positions, the first instrument processor 215 may be configured to allow the flow of energy from the power source 260 to the motor 245 at a level corresponding to the position of the switch 250 between the first and second positions, allowing the first surgical instrument 220 to operate at an intermediate drilling or cutting speed. For example, if the first instrument processor 215 receives a signal that the switch sensor has detected that the switch 250 is positioned half-way (50%) between the first and second positions, the first instrument processor 215 may be configured to allow the flow of energy from the power source 260 to the motor 245 at a level that allows the first surgical instrument 220 to operate at a rate of 50% of the maximum drilling or driving speed. Alternatively, the first instrument processor 215 may be configured to allow the maximum flow of energy from the power source 260 to the motor 245 whenever the switch 250 is in a position other than the first position, allowing the first surgical instrument 220 to operate at the maximum drilling or cutting speed when the switch 250 is in the second position or any of the intermediary positions. An exemplary switch sensor may be found in U.S. Pat. No. 9,295,476, which is hereby incorporated in by reference in its entirety.

The first surgical instrument assembly 200 may also include a first alert device 255. In an exemplary configuration, the first alert device 255 may include any one of various devices such as a vibrating device that is placed in contact with the medical professional and configured to vibrate to notify the medical professional of a particular condition or to provide a warning, an audible device, such as a speaker configured to provide an audible alert to notify the medical professional of a particular condition or to provide a warning, or a visually perceivable device or indicator to provide a visual indication to notify the medical professional of a particular condition or to provide a warning. Exemplary first alert devices may be found in International Publication No. WO 2021/062373 A2, which is herein incorporated by reference in its entirety.

The first alert device 255 may be configured to be in communication with the first instrument processor 215 or the processor of the power source 260. The first instrument processor 215 or other processor may be configured to send a signal to activate the first alert device 255 to provide a warning or notification based on a pre-programmed condition or setting. For example, as described above, the medical professional may input defined conditions and/or settings into the surgical navigation system 100, such as selecting cortical walls, nerves, blood vessels, or similar anatomical structures that the medical professional wishes to avoid and establish alert zones surrounding those anatomical structures. The first instrument processor 215, based on data provided by the navigation computer 140, may be configured to send a signal to activate the first alert device 255 upon the end-effector 240 of the first surgical instrument 220 entering one of the alert zones, as defined by the medical professional. The first instrument processor 215 or other processor, based on data provided by the navigation computer 140, may also be configured to send a signal to activate the first alert device 255 upon the end-effector 240 of the first surgical instrument 220 being off trajectory and/or upon the end-effector 240 reaching the target location.

While the first alert device 255 is illustrated as being coupled to or proximate the switch 250 of the first surgical instrument assembly 200, it is contemplated that the first alert device 255 may be coupled to and/or positioned in alternative positions. For example, when the first alert device 255 includes a tactile device, the first alert device 255 may be configured as a vibrating member that is removably attached to the medical professional. The first alert device 255 may be configured as a wearable device, such as a bracelet to be worn on the medical professional's wrist or arm so that the medical professional would be able to feel the first alert device 255 vibrating upon the occurrence of the defined condition. Alternatively, when the first alert device 255 includes an audible device, the first alert device 255 may be configured as a speaker that is removably attached to the medical professional. The first alert device 255 may be configured as a blue-tooth speaker or earpiece to be worn on the medical professional's head or positioned within the medical professional's ear so that the medical professional would be able to hear the first alert device 255 producing a noise upon the occurrence of the defined condition.

While not required, there are a number of advantages to positioning the first alert device 255 away from the first surgical instrument 220. For example, one advantage of positioning the first alert device 255 away from the first surgical instrument 220 is that it may reduce the size of the first surgical instrument 220. This may allow for the first surgical instrument 220 to fit in smaller spaces. A smaller first surgical instrument 220 may also provide a less obstructed view of the surgical site for the medical professional. Another advantage of positioning the first alert device 255 away from the first surgical instrument 220, particularly in the case of a tactile device, is that the first alert device 255 will not vibrate or impact the movement of the first surgical instrument 220 while still providing an alert or notification to the medical professional. During highly technical procedures, an alert that vibrates the first surgical instrument 220 may be likely to cause the medical professional to move the first surgical instrument 220 in an undesirable position as a result of being startled by the first alert device 255 and/or the vibration imparting an undesirable movement to the first surgical instrument 220.

The first surgical instrument assembly 200 may also include a tracking device 230. The tracking device 230 may be coupled to the handpiece 225 of the first surgical instrument 220. The tracking device 230 may include a plurality of markers 235 that are identifiable by the tracking unit 110 of the surgical navigation system 100. The markers 235 may include passive tracking elements (e.g., reflectors) for transmitting light signals (e.g., reflecting light emitted from the tracking unit 110) to the sensors 115. In other configurations, the markers 235 may be configured as active tracking markers. It is also contemplated that the markers 235 may include a combination of active and passive arrangements.

The markers 235 may be arranged in a defined or known position and orientation relative to the other markers 235 in order to allow the surgical navigation system 100 to determine the position and orientation (pose) of the surgical instrument 220. For example, the markers 235 may be registered to the first surgical instrument 220 to allow the surgical navigation system 100 to determine the position and/or orientation of an end-effector 240 or cutting portion of the first surgical instrument 220 within a defined space, such as the surgical field. In one exemplary configuration, the surgical navigation system 100 may be configured to determine the position and/or orientation of the end-effector 240 or cutting portion of the second surgical instrument 220 or relative to the target trajectory and/or the target location of the planned surgical pathway. In another exemplary configuration, the surgical navigation system 100 may also be configured to determine the position and/or orientation of the end-effector 240 or cutting portion of the second surgical instrument 220 or relative to critical anatomical structures within the patient's body, as well as relative to the virtual boundaries and/or alert zones.

The surgical system 10 may alternatively include a second surgical instrument assembly 300 to be used with the surgical navigation system 100. For example, the second surgical instrument assembly 300 may include a second surgical instrument 320, such as a high-speed surgical bur or ultrasonic surgical handpiece, including a handpiece 325. The handpiece 325 may be coupled to a console 310 that is configured to control the operation of various components of the second surgical instrument 320. The handpiece 325 may be shaped to define a handle or grip portion for the medical professional to hold while performing a medical procedure. Exemplary second surgical instruments that connect to consoles may be found in U.S. Pat. No. 10,016,209 and U.S. Patent Publication No. 2019/0117322, which are each hereby incorporated by reference in their entirety.

The second surgical instrument 320 may further include a second instrument processor 315 and a motor 345. The second instrument processor 315 may be disposed within the console 310 of the second surgical instrument assembly 300. The motor 345 may be disposed within the handpiece 325 of the second surgical instrument 320. The second instrument processor 315 and the motor 345 may be in communication with one another and the second instrument processor 315 may be configured to control the operation of the motor 345, and by extension the second surgical instrument 320. For example, the second surgical instrument 320 may be coupled to the console 310 by a cord connecting the second instrument processor 315 to the motor 345 to allow communication between the second instrument processor 315 to the motor 345 to control operation of the motor 345. The second instrument processor 315 may also include an end-effector 340, such as a high-speed cutting bur or ultrasonic tip. The end-effector 340 may be coupled to the handpiece 325 of the second surgical instrument 320 such that the motor 345 may be operably coupled to the end-effector 340. For example, the motor 345 may be configured to actuate the high-speed cutting bur 340 to grind and/or remove biological tissue from the surgical site or to vibrate the ultrasonic tip. The second instrument processor 315 may be in communication with the motor 345 and configured to control the operation of the motor 345, and by extension the high-speed cutting bur 340.

The second surgical instrument assembly 300 may also include a tracking device 330. The tracking device 330 may be coupled to the handpiece 325 of the second surgical instrument 320. The tracking device 330 be similar to as described above for the first surgical instrument assembly 200. For instance, the tracking device 330 may include a plurality of markers 335 that are identifiable by the tracking unit 110 of the surgical navigation system 100, and each the markers 335 may be arranged in a defined or known position and orientation relative to the other markers 335 in order to allow the surgical navigation system 100 to determine the position and orientation (pose) of the second surgical instrument 320. The second instrument processor 315 may also be in communication with the navigation computer 140 and configured to exchange data related to the position and/or orientation of the second surgical instrument 320, as well as data related to the operation of the second surgical instrument 320. For example, the second instrument processor 315 and the navigation computer 140 may be configured to communicate data between one another related to the operation of the second surgical instrument 320 based on the position and/or orientation of the second surgical instrument 320 as detected by the surgical navigation system 100. It is also contemplated that additional surgical instruments may be coupled to the console 310 and/or in communication with the second instrument processor 315 disposed within the console 310.

The second surgical instrument assembly 300 may also include a power source (not shown). The power source may be coupled to the console 310 of the second surgical instrument assembly 300 and configured to provide energy to the motor 345 of the second surgical instrument 320 to actuate the end-effector 340. It is also contemplated that the console 310 may include a cord configured to be plugged into an outlet that is connected to an electrical grid for supplying energy to the second surgical instrument assembly 300. The power source may be in electrical communication with the second instrument processor 315 and/or the motor 345 and configured to selectively provide power to the motor 345 to actuate the end-effector 340.

The second surgical instrument assembly 300 may also include a switch 350, such as a footswitch, trigger or button, that is operably coupled to the second instrument processor 315. The switch 350 may be configured to produce and/or communicate a signal to the second instrument processor 315 based on a user input to control the operation of the second surgical instrument 320. While not illustrated in the FIGS., it is contemplated that a plurality of surgical instruments 320 may be coupled to the console 310 and controlled by a footswitch. The switch 350, such as a footswitch, may be configured to control each of the plurality of surgical instruments 320. For example, a single footswitch may include a plurality of buttons, each of which may be assigned to one of the plurality of surgical instruments 320. An exemplary surgical system including a switch connected to a console for controlling a plurality of surgical instruments is disclosed in U.S. Pat. No. 10,820,912, which is incorporated in its entirety.

The second surgical instrument assembly 300 may also include a second alert device 355 similar to that of the first alert device 255. The second alert device 355 may include one of the audible, tactile, and/or visually perceptible devices discussed with respect to the first alert device 255. The second alert device 355 may be configured to be in communication with the second instrument processor 315 or directly with the navigation processor. The second instrument processor 315 or navigation processor may be configured to send a signal to activate the second alert device 355 to provide a warning or notification based on a pre-programmed condition or setting. The second alert device 355 as illustrated as being coupled to the switch 350 of the second surgical instrument assembly 300, it is contemplated that the second alert device 355 may be coupled to and/or positioned in alternative positions.

The surgical system 10 may include a third surgical instrument assembly 400 in communication with the surgical navigation system 100. For example, the third surgical instrument assembly 400 may include a third surgical instrument 420, such as an ultrasonic instrument, including a handpiece 425. The handpiece 425 may be coupled to a console 410 that is configured to control the operation of various components of the third surgical instrument 420. The handpiece 425 may be shaped to include a handle or grip portion for the medical professional to hold while performing a medical procedure.

The third surgical instrument 420 may further include a third instrument processor 415 and a motor 445. The third instrument processor 415 may be disposed within the console 410 of the third surgical instrument assembly 400. The motor 445 may be disposed within the handpiece 425 of the third surgical instrument 420. The third instrument processor 415 and the motor 445 may be in communication with one another. The motor 445 may include a piezoelectric element configured to expand and contract upon the application of an electric current to the piezoelectric element. The piezoelectric element may include a plurality of disc-shaped piezoelectric elements arranged end to end in a stack. The third instrument processor 415 may be configured to control the operation of the motor 445, and by extension the third surgical instrument 420. For example, the third surgical instrument 420 may include an end-effector 440, such as an ultrasonic tip assembly.

The end-effector 440 may include an ultrasonic tip assembly including a horn of which an ultrasonic tip portion vibrates at an ultrasonic wave velocity as the piezoelectric element(s) expand and contract. The ultrasonic tip assembly may also include an external sheath at least partially disposed over the horn except for the ultrasonic tip portion. The end-effector 440 may be coupled to the handpiece 425 of the third surgical instrument 420 such that the motor 445 may be operably coupled to the end-effector 440. For example, the motor 445 may be configured to actuate the ultrasonic tip assembly to grind and/or remove biological tissue from the surgical site. The third instrument processor 415 may be in communication with the motor 445 and configured to control the flow of electric current to the piezoelectric element(s), controlling operation of the motor 445, and by extension the ultrasonic tip assembly.

The third surgical instrument assembly 400 may also include a tracking device 430. The tracking device 430 may be coupled to the handpiece 425 of the third surgical instrument 420. The tracking device 430 may be similar as defined above for the other instrument assemblies. For instance, the tracking device 430 may include a plurality of markers 435 that are identifiable by the tracking unit 110 of the surgical navigation system 100, and each the markers 435 may be arranged in a defined or known position and orientation relative to the other markers 435 in order to allow the surgical navigation system 100 to determine the position and orientation (pose) of the third surgical instrument 420. The third instrument processor 415 may also be in communication with the navigation computer 140 and configured to exchange data related to the position and/or orientation of the third surgical instrument 420, as well as data related to the operation of the third surgical instrument 420. For example, the third instrument processor 415 and the navigation computer 140 may be configured to communicate data between one another related to the operation of the third surgical instrument 420 based on the position and/or orientation of the third surgical instrument 420 as detected by the surgical navigation system 100.

The third surgical instrument assembly 400 may also include a power source (not shown). The power source may be coupled to the console 410 of the third surgical instrument assembly 400 and configured to provide energy to the motor 445 of the third surgical instrument 420 to actuate the end-effector 440. For example, the power source may include a removable battery pack. It is also contemplated that the console 410 may include a cord configured to be plugged into an outlet that is connected to an electrical grid for supplying energy to the third surgical instrument assembly 400. The power source may be in electrical communication with the third instrument processor 415 and/or the motor 445 and configured to selectively provide power to the motor 445 to actuate the end-effector 440.

The third surgical instrument assembly 400 may also include a switch 450, such as a footswitch, pedal or button, that is operably coupled to the third instrument processor 415. The switch 450 may be configured to produce and/or communicate a signal to the third instrument processor 415 based on a user input to control the operation of the third surgical instrument 420.

The third surgical instrument assembly 400 may also include a third alert device 455 that is similar to that of the first alert device 255 and the second alert device 355. The third alert device 455 may include one of the audible, tactile, and/or visually perceptible devices discussed with respect to the first alert device 255. The third alert device 455 may be configured to be in communication with the third instrument processor 415. The third instrument processor 415 may be configured to send a signal to activate the third alert device 455 to provide a warning or notification based on a pre-programmed condition or setting.

The surgical instrument assemblies 200, 300, 400 described above are intended to be exemplary instruments and/or configurations within the surgical system 10 but are not intended to be limiting. Other types and forms of surgical instrument assemblies are contemplated. While a plurality of exemplary surgical instrument assemblies 200, 300, 400 are described as being a part of the surgical system 10 and in communication with the surgical navigation system 100, it is contemplated that the surgical system 10 may only include a single surgical instrument assembly 200, 300, 400. Furthermore, while the surgical system 10 illustrated in FIG. 2 includes three surgical instrument assemblies 200, 300, 400 and a single surgical navigation system 100, it is contemplated that the surgical system 10 may be configured to include any combination of surgical instrument assemblies 200, 300, 400, and/or surgical navigation systems 100. For example, the surgical system 10 may include a single surgical instrument assembly 200, 300, 400 and a plurality of surgical navigation systems 100.

Referring to FIG. 4, an exemplary configuration of a GUI 150A of the surgical navigation system 100 is illustrated. The GUI 150A may be configured as a touch screen on the display unit 120 of the surgical navigation system 100. As shown in FIG. 4, the GUI 150A may be referred to as a segmentation interface. The GUI 150A may include a select region button 141 for selecting a region to be segmented and a segment button 142 which the medical professional selects to segment the selected region.

The surgical navigation system 100 may be configured to utilize segmentation to facilitate alert zone planning for generating alerts or controlling a parameter of the surgical instruments based on the tracked pose thereof as described in greater detail below. The medical professional may provide input to the user input devices 130 by selecting the select region button 141 of the graphic user interface (GUI) 150A to define a region of interest, such as a lumbar vertebra region 153, of the patient images when the operation is targeted at the lumbar vertebrae and thus the segmentation of the patient images may be limited to the lumbar vertebra region 153. Once the medical professional has selected the region of interest, such as the lumbar vertebra region 153, the medical professional may select a segment button 142 to segment the lumbar vertebra region 153.

The segmentation may be performed automatically, semi-automatically, or manually. Once such automatic segmentation that the surgical navigation system 100 may be configured to perform is an atlas-to-image mapping process to map a three-dimensional vertebra model to each of the vertebra of the lumbar vertebra region 153. The three-dimensional vertebra model may be overlaid onto each vertebra of the lumbar vertebra region 153 and then the vertebra model may be automatically, manually, or semi-automatically deformed until the three-dimensional vertebra model is adapted to each vertebra of the lumbar vertebra region 153.

In an example of an automatic segmentation process, the surgical navigation system 100 may be configured to use a model fitting algorithm to perform the segmentation of the lumbar vertebra region 153. The model may, for example, represent variation within a set of images of a structure represented in the first image and may be fitted to the first image based upon properties of the image. The fitting may include applying a fitting technique selected from the group consisting of: rigid registration, non-rigid registration, active shape modelling and active appearance modelling. The fitting technique may be similar to that described by International Publication No. WO/2011/098752 A2, which is herein incorporated by reference. Indeed, the fitting may include applying any suitable fitting technique. While the example is provided that the automatic segmentation may be performed based on a model fitting algorithm, the system may be configured to use another suitable algorithm for performing automatic or semi-automatic segmentation.

In a semi-automatic segmentation process, the medical professional may be required to provide some sort of input, such as to identify an anatomical landmark, during the segmentation process. The surgical navigation system 100 may be configured to implement one of the semi-automatic segmentation methods for segmenting the lumbar vertebra region 153 based on one of the methods described in U.S. Pat. No. 8,698,795 (entitled, “Interactive Image Segmentation), the contents which are hereby incorporated by reference.

The surgical navigation system 100 may also be configured to use a combination of manual, semi-automatic, and automatic segmentation algorithms to perform the segmentation of the lumbar vertebra region 153. For example, the surgical navigation system 100 may perform an initial segmentation using a first algorithm, such as with the model fitting algorithm described above and then refine the initial segmentation of one or more vertebra of the lumbar vertebra region 153 using a second segmentation algorithm, such as with a graph cuts algorithm. A method for segmenting a medical image based on a first segmentation algorithm and second segmentation algorithm is described in U.S. Patent Publication No. 2021/0192743A1, which is herein incorporated by reference in its entirety.

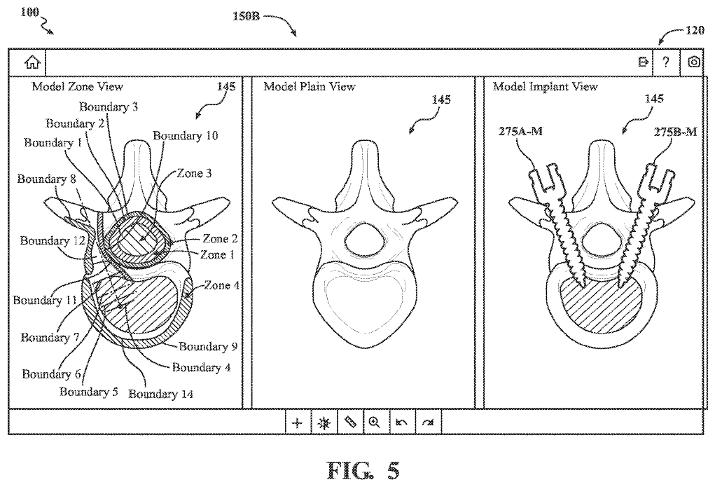

During or after, the segmentation of the lumbar vertebra region 153, the surgical navigation system 100 may map one or more virtual implants, a plurality of alert zones, and/or a plurality of virtual boundary to each vertebra of the lumbar vertebrae region 153 based on a three-dimensional vertebra model. With reference to FIG. 5, the graphic user interface 150B shows various views of a vertebra model 145, the various views including an implant view in which a superior view of a vertebra model 145 is shown with model implants 275A-M, 275B-M in optimal poses, a model alert zone view of the vertebra model 145 in which a plurality of model alert zones and virtual boundaries are shown in a superior view, the plurality of model alert zones (Zone 1-6) defined by a plurality of model virtual boundaries (Boundary 1-13), and a plain model view of the vertebra model 145 in which the model is shown with no model implants or no model alert zones. The vertebra model 145 may be defined in a second coordinate system (i.e., a vertebral body coordinate system). The optimal pose for the model implants 275A-M, 275B-M are defined in the second coordinate system with respect to various landmarks or anatomical features of the vertebra model 145.

With reference to FIG. 5, in the model alert zone view, a pose of each of the plurality of model alert zones and/or virtual boundaries is shown relative to the vertebra model 145 and defined in the second coordinate system. The plurality of model alert zones includes a first model alert zone (Zone 1), a second model alert zone (Zone 2), a third model alert zone (Zone 3), a fourth model alert zone (Zone 4), a fifth model alert zone (Zone 5), and a sixth model alert zone (Zone 6). Model alert zone 1 may be defined relative to the spinal cord. For example, model alert zone 1 may be defined as a volume between a first virtual boundary (Boundary 1) and a second virtual boundary (Boundary 2). Boundary 1 may be defined relative to an outer perimeter of the spinal cord. Boundary 2 may be placed at a default distance from Boundary 1 based, at least in part, on the surgical procedure being performed. For example, Boundary 2 may be offset by Boundary 1 by two millimeters.

Model alert zone 2 may be defined a second distance from the critical anatomical structure, such that the second distance is greater than the first distance. Model alert zone 2 may be defined as a volume between by Boundary 2 and a third virtual boundary, Boundary 3. Boundary 3 may be placed at a default distance from Boundary 2 based, at least in part, on the surgical procedure being performed. Model alert zone 3 may be defined at the boundary of and/or including the critical anatomical structure. For example, Boundary 3, may define the outer perimeter of model alert zone 3. An eighth boundary, Boundary 8, and a ninth boundary, Boundary 9, define an outer perimeter of the vertebra. Model alert zone 4 may be defined relative to an outer perimeter of the vertebra. Model alert zone 4 may be bound by a fourteenth virtual boundary, Boundary 14, and a ninth virtual boundary, Boundary 9. Boundary 14 may be offset from Boundary 9 which may be defined at the outer perimeter of the vertebra.

Model alert zone 5 may be defined such that it is contoured around a critical anatomical structure (the central foramen within the vertebra to alert the medical professional when the end-effector 240 of the first surgical instrument 220 is approaching the critical anatomical structure to prevent the medical professional from contacting the critical anatomical structure. Model alert zone 5 may be defined by a tenth virtual boundary, Boundary 10, and an eleventh virtual boundary, Boundary 11. Boundary 10 may touch or be directly adjacent to Boundary 3 with Boundary 11 placed at a default distance from Boundary 10.

Model alert zone 6 is curved around the outer perimeter of the pedicle to alert the medical professional when an end-effector 240 of the first surgical instrument 220 may be approaching the outer perimeter of the vertebra to prevent the medical professional from breaching the outer perimeter of the pedicle. Model alert zone 6 may be defined by a twelfth virtual boundary, Boundary 12, and Boundary 8. Boundary 12 may be offset from an outer boundary of the pedicle region of the vertebra, while Boundary 8 may be defined at the outer perimeter of the pedicle region of the vertebra.

It is also contemplated that the surgical navigation system 100 may be configured to define the virtual boundaries (Boundary 1-13) and/or model alert zones (Zone 1-6) based on information selected or input by the medical professional. For example, the surgical navigation system 100 may be configured to define the model alert zones (Zone 1-6) based on one or more of the following items input by the medical professional: the type procedure to be performed, the location of the procedure on the patient, the type of implant 275 to be used, the type of surgical instrument 220, 320, 420 to be used and/or the type end-effector 240, 340, 440. The medical professional may then have the opportunity to modify or alter the virtual boundaries (Boundary 1-13) and/or model alert zones (Zone 1-6) of the three-dimensional vertebra model using the user input device 130 and/or the GUI 150.

In addition to the plurality of model alert zones (Zone 1-6) and the virtual boundaries (Boundary 1-13), the vertebra model 145 may include a plurality of features or areas defined with respect to the second coordinate system. The plurality of features or areas may include a superior articular surface, spinous process, mammillary process, inverse process, pedicle, vertebra foramen, superior articular process, vertebral arch, superior vertebral notch, vertebral body, etc.

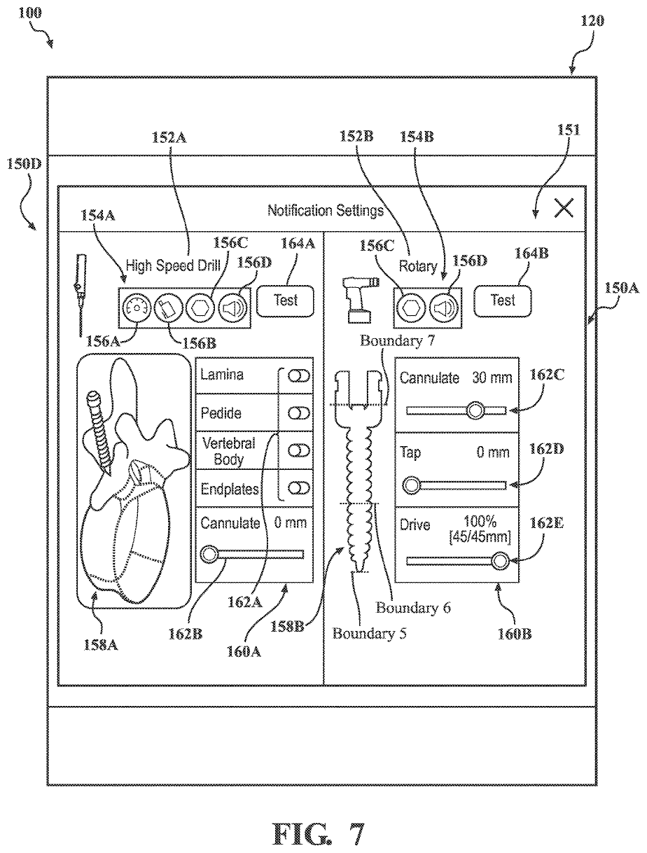

The vertebra model 145 may include multiple planar virtual boundaries that can be used to delineate multiple target depths (e.g., three target depths) for separate instruments to be used in a single procedure. For example, a fifth virtual boundary (model boundary 5) representing target depth for a drill to bore the hole, a sixth virtual boundary (model boundary 6) representing target depth for the tap, and a seventh virtual boundary (model boundary 7) representing target depth for the driver to insert the screw, as are illustrated in FIG. 5 and will be explained in greater detail below with respect to FIG. 7.

During or after segmentation, the model implants 275A-M-, 275B-M, the model alert zones (Zone 1-6), model virtual boundaries (Boundary 1-12) of the vertebra model 145, may be mapped to the vertebra of the lumbar vertebra region 153 so that a patient specific surgical plan is generated according to the vertebra model. For example, the L3 vertebra of the lumbar vertebra region 153, after the lumbar vertebra region 153 is segmented and mapped for implants, alert zones, and virtual boundaries, each vertebra will have a planned pose of one or more implants 275A, 275B, a plurality of alert zones (Zone 1-6) and a plurality of virtual boundaries (1-12). The medical professional may adjust one or more of the plurality of alert zones (Zone 1-6), virtual boundaries (Boundary 1-12) and planned implant poses 275A, 275B, as described in greater detail below.

The virtual boundaries (Boundary 1-12) and alert zones (Zone 1-6) may be one-dimensional (1D), two-dimensional (2D), three-dimensional (3D), and may include a point, line, axis, trajectory, plane (an infinite plane or plane segment bounded by the anatomy or other virtual boundary), volume or other shapes, including complex geometric shapes. The virtual boundaries (Boundary 1-12) and alert zones (Zone 1-6) may be represented by pixels, point clouds, voxels, triangulated meshes, other 2D or 3D models, combinations thereof, and the like disclosed by U.S. Patent Publication No. 2018/0333207 and U.S. Pat. No. 8,898,043, which are incorporated by reference.

As will be discussed in further detail below, the virtual boundaries and/or alert zones may be used in various ways. For example, the navigation computer 140 may control certain operations/functions of the surgical instruments 220 based on a relationship of the surgical instruments 220 to one or more virtual boundaries and/or alert zones (e.g., spatial, velocity, etc.). Other uses of the virtual boundaries are also contemplated. The plurality of alert zones (Zone 1-6) may be activated/deactivated as described in greater detail with respect to the alert interface, one at a time, by the navigation computer 140.

The surgical navigation system 100 may be programmed and/or configured to manipulate the speed of the motor 245, 345, 445 of the surgical instrument 220, 320, 420, and/or activating an alert device 255, 355, 455, upon the surgical navigation system 100 determining the surgical instrument 220, 320, 420 is at/or adjacent to one or more of the virtual boundaries or has entered one of the defined alert zones. For example, when the surgical navigation system 100 detects that that the first end-effector 240A is coupled to the handpiece 225, the surgical navigation system 100 may be configured to transmit a signal to the instrument processor 215, 315, 415 of the surgical instrument 220 to deactivate the motor 245, 345, 445 when the first end-effector 240A is adjacent and/or distal to Boundary 5.

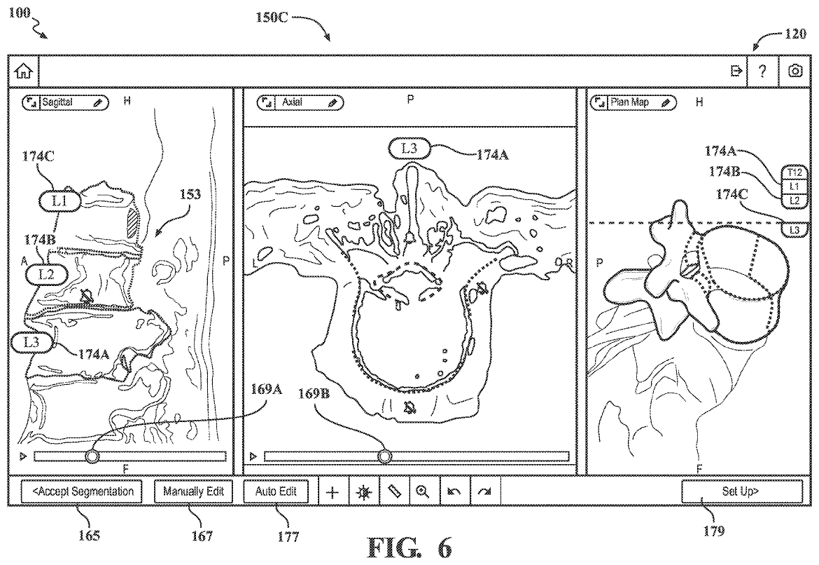

With reference to FIG. 6, the GUI 150C shows the lumbar vertebra region 153 that has been segmented according to one of the described methods for segmentation. The GUI 150C includes a sagittal view, an axial view, and a model or perspective view of the lumbar vertebrae region 153, with the L3 vertebra at the focus of the GUI 150C. The GUI 150C may include one or more buttons such as an accept segmentation button 165, a manual edit segmentation button 167, an auto-edit segmentation button 177, and a set-up button 179. The set-up button when selected by the medical professional may bring up a notifications settings interface as discussed in more detail with respect to FIG. 7. The GUI 150C may include one or more labels 174 identifying the vertebra displayed on the GUI 150C. As shown in FIG. 6, three labels 174A, 174B, 174C are shown on the GUI 150C, with a first label 174A identifying the L3 vertebra, a second label 174B identifying the L2 vertebra adjacent to the L3 vertebra, and a third label 174C identifying the L1 vertebra adjacent to the L2 vertebra. The GUI 150C may be configured such that the user may manipulate the GUI 150C to navigate between the various vertebrae such as the L3 vertebra, the L2 vertebra, and the L1 vertebra. The navigation computer 140 may be configured to display the L3 vertebra in the center of the GUI 150C when the label corresponding to the L3 is selected. Alternatively, the medical professional may select the second label 174B, and the navigation computer 140 may be configured to identify the L2 vertebra proximate the second label 174B as the primary anatomical structure, and position the L2 vertebra and the second label 174B in the center of the GUI 150C. While only three labels are shown in FIG. 6, the GUI 150C may be configured to include any number of labels 174.

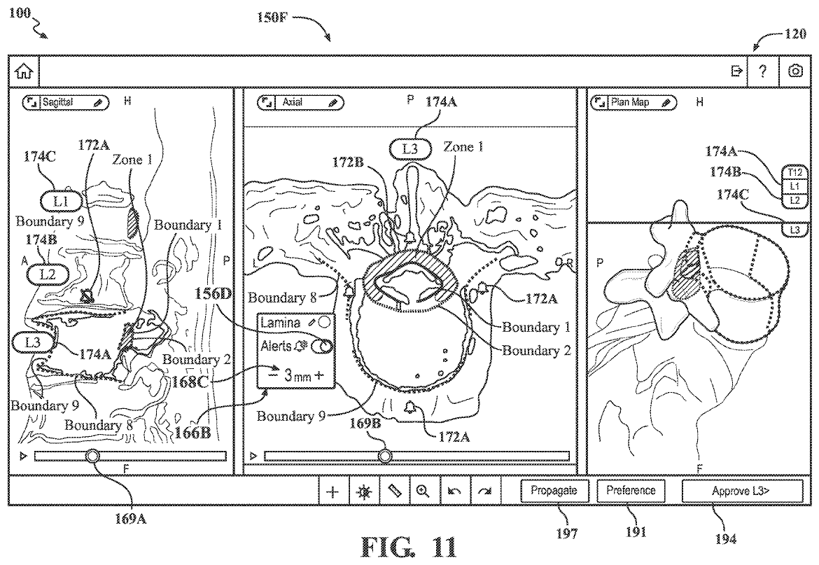

The navigation computer 140 may be configured such that when the medical professional selects the propagate button 197, the navigation computer will propagate the alert zone edits to all vertebra associated with a level label 174 that is set to the first illumination state. As such, the medical professional may deselect a level label 174 prior to selecting the propagate button 197 to exclude a vertebra from propagation of alert zone edits made to a particular vertebra. In FIG. 11, all level labels 174 have been selected so the navigation computer 140 will propagate the alert zone edits made to the L3 vertebra to the L1 vertebra and the L2 vertebra. However, if for example, the medical professional deselected level label 174C associated with the L1 vertebra, the navigation computer would only propagate the alert zone edits to the L2 vertebra.

Referring to the GUI 150C, the lumbar vertebrae region 153 is shown after segmentation has been performed with the L3 vertebra displayed by the GUI 150C, including the location of the various virtual boundaries (1, 8, 9) defining the segmentation of the L3 vertebra. If the medical professional is not satisfied with the segmentation, the medical professional may select the manual edit segmentation button 167 and then reposition one or more of the virtual boundaries to a desired position or may select the auto-edit segmentation button 177 and then select a region of the L3 vertebra for refined segmentation by a second segmentation algorithm as disclosed by U.S. Patent Publication Number 2021/0192743A1, which was previously incorporated by reference in its entirety.

The medical professional may navigate through the selected lumbar vertebra region 153 to shift focus of the GUI 150C within the selected lumbar vertebra region 153 by interacting with the slider bars 169A, 169B. Once the medical professional has reviewed a segmented vertebra, such as the segmented L3, the medical professional may choose to select the accept segmentation button 165 to signal to the navigation computer 140 that the medical professional is satisfied with the segmentation of the L3 vertebra and may then review the other remaining segmented vertebra within the lumbar vertebra region 153 for accuracy.

Referring to FIG. 7, an exemplary configuration of a GUI 150F of the surgical navigation system 100 is illustrated, referred to as a notification interface 151. The GUI 150F may include a plurality of buttons 156, 162, 164, 169 configured to receive input by the medical professional to control or modify or adjust the various settings for the alerts to be provided during the execution of a medical procedure, as will be discussed in greater detail below.

The notification interface 151 may include a tool selection button 152A, 152B. The tool selection button 152A, 152B may allow the medical professional to select the surgical instrument assembly 200, 300, 400 from a populated list of surgical instruments, or may allow the medical professional to input a specific surgical instrument assembly 200, 300, 400 that will be utilized during the surgical procedure. For example, the tool selection button 152A, 152B may allow the medical professional to select the second surgical instrument 320 including a high-speed cutting bur. This identifies the particular surgical instrument 320 to the navigation computer 140, allowing the navigation computer 140 to populate the various virtual boundaries and/or alert zones that will be utilized for the identified instrument. The tool selection button 152A, 152B may also be configured to allow the medical professional to select the surgical instrument assembly 200, 300, 400, as well as one or more end-effectors 240, 340, 440 that may be coupled to the surgical instrument 220, 320, 420. For example, the medical professional may select the first surgical instrument 220, and further selected one or more of the end-effectors 240A, 240B, 240C that may be utilized during procedure to allow the navigation system to populate the various virtual boundaries and/or alert zones for each of the various end-effectors 240A, 240B, 240C.

The notification interface 151 may also include one or more alert buttons 156A, 156B, 156C, 156D that are utilized to manipulate the various alerts described above. A first alert button 156A may be configured to allow the medical professional to activate or deactivate an alert related to the rotational speed of the end-effector 240, 340, 440. For example, as described above, the navigation computer 140 and/or the instrument processor 215, 315, 415 may be configured to manipulate the rotational speed (RPM's) of the end-effector 240, 340, 440 based on the position of the end-effector 240, 340, 440 relative to at least one of the virtual boundaries and/or at least one of the alert zones. A second alert button 156B may be configured to allow the medical professional to activate or deactivate a tactile alert. For example, the medical professional may manipulate the second alert button 156B to activate one of tactile alerts described above. This may include the navigation computer 140 being configured to send a signal to the surgical instrument assembly 200, 300, 400 to activate the alert device 255, 355, 455 configured to provide a tactile alert to the medical professional based on the position of the end-effector 240, 340, 440 relative to at least one of the virtual boundaries and/or at least one of the alert zones.

A third alert button 156C may be configured to allow the medical professional to activate or deactivate a visual alert. For example, the medical professional may manipulate the third alert button 156C to activate one of the visual alerts. This may include the navigation computer 140 being configured to send a signal to the surgical instrument assembly 200, 300, 400 to activate the alert device 255, 355, 455 configured to provide a visual alert to the medical professional based on the position of the end-effector 240, 340, 440 relative to at least one of the virtual boundaries and/or at least one of the alert zones. A fourth alert buttons 156D may be configured to allow the medical professional to activate or deactivate one of the audible alerts described above. For example, the medical professional may manipulate the fourth alert button 156D to activate the audible alert, such that the navigation computer 140 may send a signal to the surgical instrument assembly 200, 300, 400 to activate the alert device 255, 355, 455 configured to provide an audible alert to the medical professional based on the position of the end-effector 240, 340, 440 relative to at least one of the virtual boundaries and/or at least one of the alert zones.

The notification interface 151 of the GUI 150D may also include one or more alert graphics 158A, 158B. The alert graphic(s) 158A, 158B may be specific to the particular surgical instrument and/or end-effector and may be configured to provide a schematic and/or visual representation of the location of the various virtual boundaries and/or alert zones described in greater detail below. The first alert graphic 158A may include a visual representation of the surgical region and any implants or devices to be inserted during the medical procedure to assist the medical professional in identifying the location of the procedure and with setting the various alerts. For example, as shown in FIG. 7, the first alert graphic includes a visual of the vertebral body with the region where the procedure will take place outlined in dotted lines. The first alert graphic 158A may also include a visual of the pedicle screw to be inserted during the procedure.

The second alert graphic 158B may be configured to provide a visual representation of the implant or device to be inserted during the procedure along with markers indicating the various virtual boundaries (Boundary 5, 6, 7) relative to the implant or device to assist the medical professional in adjusting or modifying the location where the alerts assigned to each of the various virtual boundaries and/or alert zones should be triggered. For example, as illustrated in FIG. 7, the second alert graphic 158B includes a visual representation of the pedicle screw to be inserted and markers along the pedicle screw indicating the location of the various virtual boundaries (Boundary 5, 6, 7) relative to the pedicle screw that will trigger the various alerts during the procedure.

The notification interface 151 of the GUI 150D may also include an alert zone or virtual boundary setup interface 160A, 160B. The alert zone or boundary setup interface 160A, 160B may include one more prompts or buttons 162A, 162B, 162C, 162D, 162E for setting and/or manipulating when the virtual boundary will trigger one or more of the various alerts described above. A first alert zone or boundary setup interface 160A may include a first set of buttons 162A may be configured to identify the implant(s) and/or device(s) to be inserted during the procedure. This allows the surgical navigation system 100 to determine which and how many virtual boundaries and/or alert zones to provide. For example, if the medical professional manipulates the lamina button of the first set of buttons 162A to indicate a Laminotomy will be performed, the surgical navigation system 100 knows that this involves resection of various portions of one or more vertebra and the surgical navigation system 100 will identify and provide the various alert zones around the critical structures of the subject vertebra to assist the medical professional in executing the procedure. If the medical professional manipulates the pedicle button of the first set of buttons 162A to indicate a pedicle screw procedure will be executed, the surgical navigation system 100 will identify and provide the various virtual boundaries needed to assist the medical professional, in drilling, tapping, and placing the pedicle screw.

A second button 162B of the alert zone or virtual boundary setup interface 160A may correspond to a depth button. The depth button may be configured to allow the medical professional to select the depth of the alert zone for a resection procedure, such as a Laminotomy. For example, the first alert zone or virtual boundary setup interface 160A illustrated in FIG. 7 indicates that the medical professional is setting alerts for a Laminotomy based on the manipulation of the first button 162A. Based on this selection by the medical professional, the second button 162B provides a manipulatable button configured to allow the medical professional to select the depth of the alert zone to be utilized by the surgical navigation system 100 to trigger one or more of the various alerts.

A second alert zone or virtual boundary setup interface 160B of the notification interface 151 may include additional buttons 162C, 162D, 162E related to the configuration of the various virtual boundaries and/or alert zones for implants, such as screws, and triggering the alerts related to the implants during the medical procedure. For example, the second alert zone or virtual boundary setup interface 160B may be configured to provide buttons 162C, 162D, 162E for manipulating the setting of the alerts for the procedure of inserting a pedicle screw.

A third button 162C of the second alert zone or virtual boundary setup interface 160B may be configured to set distance or depth for a reference location to position the virtual boundary, such as Boundary 5, along the target trajectory. For example, as indicated in FIG. 7, the third button 162C includes a toggle to allow the medical professional to set the depth for inserting the first end-effector (i.e. a drill) before the alert is triggered. In the example, the medical professional has set the third button 162C to 30 mm, indicating the surgical navigation system 100 should trigger the alert for the first end-effector when it has reached a depth of 30 mm.

The second alert zone or virtual boundary setup interface 160B may include additional buttons 162D, 162E for manipulation and/or adjustment of when the alerts should be triggered for the second end-effector (i.e. the tap) and/or the third end-effector (i.e. the driver) for inserting the screw. As described above, the surgical navigation system 100 may be configured such that the fourth and fifth buttons 162D, 162E for manipulating the alerts for the second and third end-effectors may manipulate the location of the virtual boundary for triggering the alert based on the virtual boundary for triggering the alert for the first end-effector. For example, as indicated by the fourth button 162D, the virtual boundary for triggering the alert for the second end effector (i.e. the tap) should be shifted zero millimeters (0-mm) relative to the virtual boundary for triggering the alert for the first end-effector. However, the fourth button 162D may be manipulated to shift the virtual boundary for triggering the alert for the second end effector as needed. Similarly the fifth button 162E may be manipulated to modify or adjust the virtual boundary for triggering the alert for the third end-effector.

The notification interface 151 of the graphical user interface (GUI) 150D may also comprise an alert test button 164A, 164B. The alert test button 164A, 164B may be configured to test and/or confirm the selected alerts are active and working properly. For example, in operation, after the medical professional has selected or entered all of the various information related to the medical procedure into the notification interface 151, the medical professional may select the alert test button 164A, 164B to confirm the selected alerts are active. For example, if the medical professional selected the first alert button 156A directed to the motor speed alert to be active, the medical professional may activate the surgical instrument 220, 320, 420 and press the alert test button 164A, 164B. Pressing the alert test button 164 A, 164B instructs the navigation system to send a test signal to the instrument processor 215, 315, 415 activate the alert associated with the first alert button 156A, such as reducing the speed of the motor and by extension the speed of ration of the end-effector 240, 340, 440. Upon user selecting the alert test button 164A, 164B, each of the various alerts that have been activated based on the manipulation of an alert button 156A, 156B, 156C, 156D should be triggered. Any activated alerts that are not triggered upon selection of the alert test button 164A, 164B should be further evaluated by the medical professional to confirm they are in fact working properly prior to beginning the medical procedure.

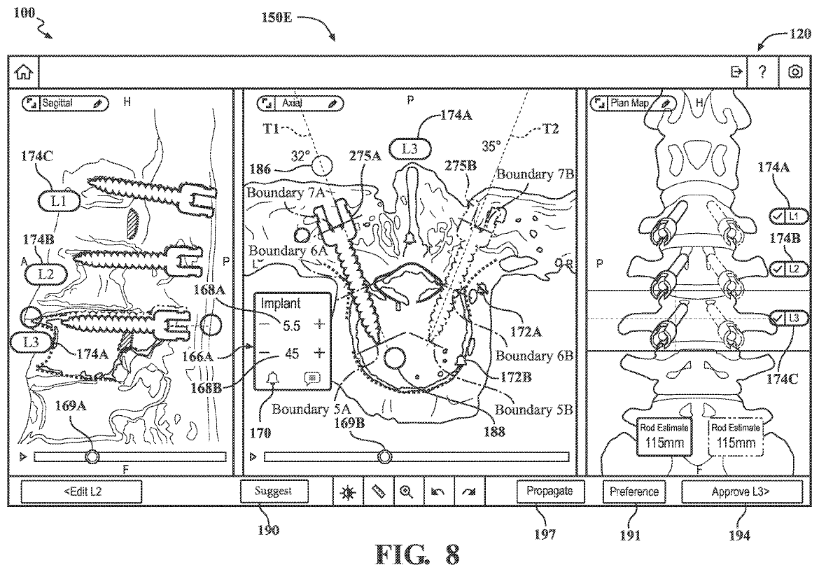

Referring to FIG. 8, once the medical professional is satisfied with the segmentation of the lumbar vertebra region 153 and has inputted various preferences to the notification interface 151, the medical professional may proceed to the implant planning step. An exemplary GUI 150E is shown in FIG. 7 for facilitating implant planning. The GUI 150E may be configured to display a visual representation of the surgical plan which includes a planned pose of the implant 275A, 275B within an image coordinate system (i.e., the first coordinate system). The medical professional may input a planned pose of the implants 275A, 275B or in some instances, such as when a medical professional has selected a suggest implant button 190, the surgical navigation system 100 may suggest a planned pose for the implants 275A, 275B obtained from the vertebra model 145. The GUI 150E may also include a preference adjustment button 191 that the medical professional may select to apply historical preferences of corrections of previous similar procedures, and a propagate button 197 that the medical professional may select to propagate corrections made to a particular vertebra, such as the L3 vertebra, to another vertebra, as will be discussed in greater detail below.

As shown, the implant 275A, 275B may define a target axis Axis-T1, Axis-T2. The surgical navigation system 100 may provide virtual boundaries (Boundary 5, 6, 7) along the target axis Axis-T1, Axis-T2 representing target depths for each of the various end-effectors 240 A, 240B, 240C utilized in execution of the procedure. As illustrated in FIG. 8, the fifth boundary (Boundary 5A, 5B are shown along the target axis Axis-T1, Axis-T2 for each of the implants 275A, 275B. The navigation computer 140 may be configured to define the fifth boundary (Boundary 5A, 5B) based on the target depth set for the tip of a first end-effector 240A and the vertebra model 145, such as a drill for boring the hole for placement of a screw 275A, 275B.

The surgical navigation system 100 may be further configured to define the sixth virtual boundary (Boundary 6A, 6B) based on the target depth for a second end-effector 240C, such as a tap for cutting threads in the hole. It is contemplated that the surgical navigation system 100 may define the sixth virtual boundary (Boundary 6A, 6B) relative to the fifth virtual boundary (Boundary 5A, 5B) based, at least in part, on the selected implant 275A, 275B, its pose and the vertebra model 145. For example, the surgical navigation system 100 may define the fifth virtual boundary (Boundary 5A, 5B) within the first coordinate system along the target axis Axis-T1, Axis-T2. Then based on the selected implant 275A, 275B, the surgical navigation system 100 may be configured to define the sixth virtual boundary (Boundary 6A, 6B) at distance from the fifth virtual boundary (Boundary 5A, 5B) based on the selected implant 275A, 275B. The navigation computer 140 may be further configured to define the seventh virtual boundary (Boundary 7A, 7B) based on the target depth for a third end-effector 240C, such as a driver for placing the screw 275A, 275B in the hole.

It is contemplated that the surgical navigation system 100 may define the seventh virtual boundary (Boundary 7A, 7B) relative to the fifth virtual boundary (Boundary 5A, 5B) based, at least in part, on the selected implant 275A, 275B, its pose, and the vertebra model 145. For example, the surgical navigation system 100 may define the fifth virtual boundary (Boundary 5A, 5B) within the first coordinate system of the patient along the target axis Axis-T1, Axis-T2. Then based on the selected implant 275A, 275B, the navigation system may be configured to define the seventh virtual boundary (Boundary 7A, 7B) at distance from the fifth virtual boundary (Boundary 5A, 5B) based on the selected implant 275A, 275B. For example, the navigation computer 140 may be configured that based on the depth of the fifth virtual boundary (Boundary 5A, 5B) and the known length of the selected implant 275A, 275B and the vertebra model 145, the navigation computer 140 can determine that the seventh virtual boundary (Boundary 7A, 7B) should be spaced thirty millimeters (30 mm) along the target axis Axis-T1, Axis-T2 from the fifth virtual boundary (Boundary 5A, 5B).

While only the fifth virtual boundary (Boundary 5A, 5B), the sixth virtual boundary (Boundary 6A, 6B), and the seventh virtual boundary (Boundary 7A, 7B) are shown in FIG. 8, additional virtual boundaries are contemplated. The navigation computer 140 may be configured define and assign a virtual boundary to each of the end-effectors 240A, 240B, 240C. The location of these virtual boundaries and or when they are configured to trigger one of the various alerts described above may be manipulated and/or adjusted as the medical professional so desires.

The GUI 150D of FIG. 8 may also include a planning interface 166A including a plurality of buttons that are manipulatable by the medical professional to modify or adapt the placement of the implant 275A, 275B. For example, planning interface 166A may include one or more diameter buttons 168A that are manipulatable by the medical professional to modify the diameter of the planned screw. The planning interface 166A may also include one or more length buttons 168B that manipulatable by the medical professional to modify the length of the planned screw 275A, 275B. The planning interface 166A may also allow the medical professional to reposition the planned screw 275A, 275B by changing its pose (i.e., position and/or orientation) relative to the L3 vertebra. The GUI 150D may also display various virtual buttons 186, 188 proximate the axis-T1 and displayed along with the planning interface 166A to facilitate adjustment of the pose of the implants 275A, 275B.

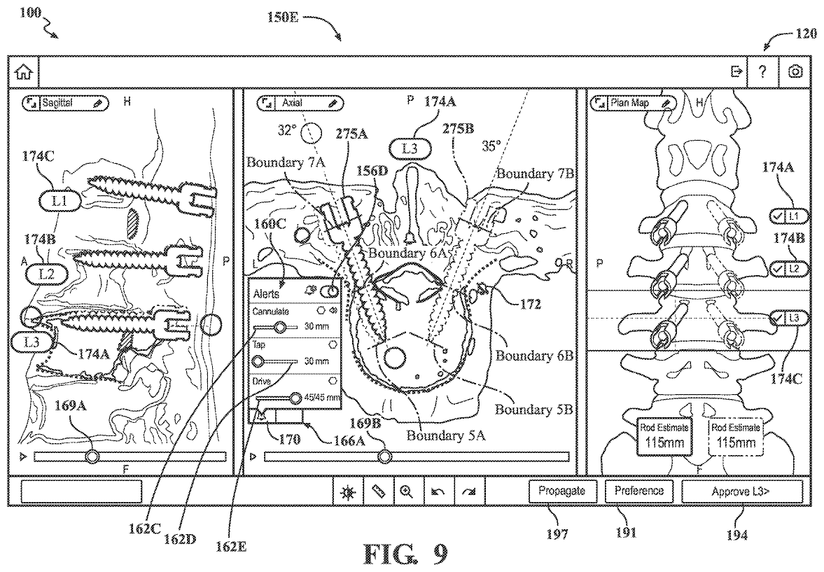

With additional reference to FIG. 9, the planning interface 166A of the GUI 150D of FIG. 8 may also include an alert button 170. The GUI 150D may be configured such that selection of the alert button 170 by the medical professional may open a boundary setup interface 160C to that the medical professional may activate, modify, and/or disable one or more the various alerts described above similar to the virtual boundary set up interface 160B described with respect to FIG. 7. The virtual boundary setup interface 160C may include additional buttons and/or prompts that are manipulatable by the medical professional to modify or adjust the virtual boundaries and/or alert zones configured to trigger one or more of the alerts. The alert indicators 172 may be positioned proximate to one or more specific virtual boundaries, such as virtual boundaries (5, 6, 7) and be configured to identify to the medical professional whether the alert assigned to one of the specific virtual boundaries (5, 6, 7) is activated, deactivated, and/or snoozed. For example, the alert indicators 172 that show a bell with a line through it, such as 172A, shown proximate to boundary 6B indicates that the nearest virtual boundaries are deactivated. Alert indicators 172 without a line through the bell, such as alert indicators 172A, indicate that the alert for the nearest virtual boundary is activated. Although not specifically illustrated, alert indicators 172 with a dashed line through the bell may indicate that the alert indicators are snoozed. The alert indicators 172 may also be selectable and/or manipulatable by the medical professional to activate or deactivate the alert assigned to the specific virtual boundaries (5, 6, 7).

The GUI 150E depicts a virtual boundary setup interface 160C for the implants 275A, 275B that may be viewed by the medical professional upon selection of the alert button 170. For example, the user selecting the alert button 170 of the planning interface 166A from the GUI 150E from FIG. 8 may cause the GUI 150E to open the virtual boundary setup interface 160C for viewing on the navigation display 120. The virtual boundary setup interface 160C may include an alert button 156D configured to allow the medical professional to activate or deactivate the various alerts. The virtual boundary setup interface 160C may also include three virtual boundary manipulation buttons 162C, 162D, 162E, one for each of the various end-effectors 240A, 240B, 240C.

As described above, the medical professional may select one of the buttons 162C, 162D, 162E to trigger an alert for each of the various end-effectors 240A, 240B, 240C. Stated differently, when the medical professional provides input to one of the buttons 162C, 162D, 162E, the corresponding virtual boundary (5, 6, 7) will be moved based on the input. Based on the value input by the medical professional using the buttons 162C, 162D, 162E, the location of the various virtual boundaries will be updated within the surgical plan utilized to navigate the system for triggering the alert(s) based on the position of the various end-effectors 240A, 240B, 240C relative to one or more virtual boundaries during the procedure.

When the navigation computer 140 has suggested a pose for implants 275A, 275B, the medical professional may need to correct or refine the pose since the navigation computer 140 is merely making a suggestion based off the vertebra model 145. As such, the medical professional may interact with one or more buttons 186, 188 positioned proximal to the target axis-T1 or target axis-T2 to reposition the implants 275A, 275B. As shown in FIG. 9, the medical professional has adjusted the pose of implant 275A so that the implant is moved away from the outer pedicle wall and toward the vertebral foramen and towards the outer virtual boundary of the L3 vertebra.

Once the medical professional is satisfied with the desired positioning of the implants 275A, 275B, the medical professional may desire to apply the corrections made to implants 275A, 275B of the L3 vertebra to other vertebra in the lumbar vertebra region 153. The medical professional may select the propagate button 197 in order for the navigation computer 140 to propagate the corrections to the remaining vertebra in the lumbar vertebra region 153. Stated differently, the navigation computer 140 may update the suggested poses of the implants of the L1 vertebra and the L2 vertebra based on the revised pose of the implants 275A, 275B of the L3 vertebra.

The navigation computer 140 may disable the navigation function discussed above with respect to the labels 174 when the medical professional selects the propagate button 197. The navigation computer 140 may be configured to when the medical professional selects the propagation button 197, prompt the medical professional to select one or more labels 174 associated with one or more desired vertebra from the lumbar vertebra region 153 to propagate the revised pose for implants 275A, 275B. In this manner, the navigation computer 140 is able to exclude a particular vertebra of the lumbar vertebra region 153 from propagation of implant corrections.

The medical professional may choose to forgo propagation of the corrections to the remining vertebra by selecting the Approve L3 vertebra button 194 which will move onto implant planning for the remaining vertebra of the lumbar vertebra region 153. When the medical professional chooses the Approve L3 vertebra button without, the default position for planned implants will corresponds to the mapped pose for the implants based on the vertebra model 145.

The navigation computer 140 may include an implant correction database which stores implant correction data and relevant patient data. The implant correction data may include spatial information, such as one or more transformations, which describe how the implant was corrected with respect to the initial suggested pose for the implant 275A, 275B or spatial information which describes how the implant was corrected relative to the model implant 275A, 275B. The implant correction data may also include implant parameters such a diameter of a screw, a length of the screw, alert zone and virtual boundary preferences (e.g., distances between virtual boundaries 5, 6, and 7).

The navigation computer 140 may access the implant corrections database to update a pose of the implants 275A-M, 275B-M of the vertebra model 145 or to suggest an implant pose for a future patient while taking into consideration the previous corrections for implant poses and preferences of the medical professional. For example, the navigation computer 140 may be configured to determine one or more transformations between the initial pose of the implant 275A and the revised pose of the implant 275A relative the first coordinate system. The navigation computer 140 may be configured to store the transformation data in the implant correction database which includes transformation data of other patients that the medical professional has previously operated on. Based on the transformation data stored in the implant correction database, the navigation computer 140 may periodically (e.g., when new patient cases entered into the implant corrections database reaches a threshold) retrieve the transformation data for the patients from the implant correction database. The navigation computer 140 may, based on the retrieved transformation data, determine an average transformation of all the transformation data stored in the implant correction database. The navigation computer 140 may update the model pose of the implant 275A-M, 275B-M, to reflect the average transformation data or other statistical analysis parameter. For example, when the number of patients reaches a threshold (e.g., one hundred patients) and the average transformation data indicates that the medical professional prefers a greater clearance between the implant and the vertebral foramen, the navigation computer 140 may update the pose of the model implants 275A-M, 275A-B to reflect the preference of the medical professional. The navigation computer may in some instances edit a suggested implant such as 275A, 275B, based on the implant correction database. In another example, if the data stored in the implant correction database indicates a clear trend of a preference for a smaller diameter screw, the navigation computer 140 may update the model implants 275A-M, 275B-M to reflect the preferences of the medical professional. The medical professional may select the preference adjustment button 191 to update the L3 vertebra based on preferences from the medical professional determined based on the implant corrections database as described above.

The navigation computer 140 may include a machine learning module that may be configured to implement a machine learning algorithm to train a machine learning model based on the implant correction database to provide a suggested pose of an implant 275A, 275B. For example, the machine learning module may train the machine learning model based on corrections, preferences, or settings that have been selected by medical professional during previous surgical procedures and the outcomes for patients. The machine learning module may train the machine learning model pursuant to one of the algorithms described by U.S. Patent Publication No. 2021/0378752A1, the contents which are hereby incorporated by reference its entirety. The navigation computer 140 may be configured to process image data for each vertebra in the lumbar vertebra region 153 and other relevant patient data through the machine learning model to provide a suggested pose for the implant 275A, 275B.

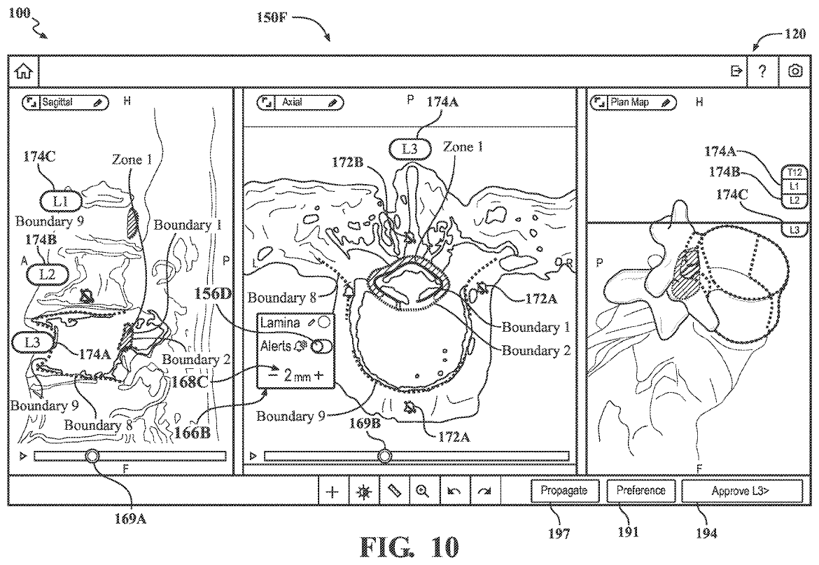

With reference to FIG. 10, an example GUI 150F is shown with multiple views of an L3 vertebra with select virtual boundaries (1, 2, 8, and 9) and alert zone 1 are shown. For ease of illustration, virtual boundaries (3-6, 7, and 10-12) and alert zones (Zone 2-6) have been omitted. However, is understood that the teachings of the disclosure as applied to zone 1 are also applicable to any of the other zones, such as zone (2-6) and virtual boundaries (Boundary 3-6, 7, and 10-12). The multiple views shown on the GUI 150 of the L3 vertebra include a sagittal view, an axial view, and a plan view described in greater detail below.

The GUI 150E may include a planning interface 166B including one or more buttons, such as an alert button 156D configured to activate and/or deactivate one or more of the various alerts. The planning interface 166B may also include a planning buttons 168C configured to allow the medical professional to manipulate the various virtual boundaries (1, 2, 8, 9) to adjust the various alert zones, such as alert zone 1, and the various alerts configured to trigger one or more the various alerts. For example, the planning buttons 168C of the planning interface 166B may be configured to receive user input to adjust the distance between one or more of the virtual boundaries (1, 2) that define at least a portion of alert zone 1 thereby increasing or decreasing the depth of one or more of the various alert zones, such as Zone 1.

The planning buttons 168C includes a pair of virtual touch buttons for the medical professional to increase or decrease the depth of one or more of the various alert zones, such as zone 1. The virtual boundaries (1, 2, 8, 9) may be provided as selectable objects that the medical professional may manipulate as the medical professional so desires. For example, the medical professional may provide input (via touchscreen or user input device) to the GUI 150F to manually manipulate the virtual boundaries (1, 2) to move one or more of the virtual boundaries (1, 2) to thereby adjust the size (e.g., depth) of the various alert zones, such as zone 1. For example, the medical professional may move virtual boundary 2 away from virtual boundary 1 to expand zone 1 or move virtual boundary 2 inward toward virtual boundary 1 to contact zone 1. In another example, the medical professional may adjust a portion of virtual boundary 1 or virtual boundary 2 to exclude or include a certain anatomical feature.

The GUI 150F may also include alert indicators 172 positioned within the display of L3 vertebra relative to the various virtual boundaries (1, 2, 8, 9) and/or zone 1. Similar to as described with respect to FIGS. 8, 9, the alert indicators 172 may be positioned proximate to a specific virtual boundaries, such as virtual boundaries (1, 2, 8, 9) and/or various alert zones, such as zone 1 and be configured to identify to the medical professional whether the alert assigned to one of the specific virtual boundaries (1, 2, 8, 9) and/or the zone 1 proximate the alert button is activated, deactivated, and/or snoozed. For example, the alert indicators that show a bell with a line through it, such as 172A, shown proximate to virtual boundaries (boundary 8, 9,) indicate that the nearest virtual boundaries are deactivated. Alert indicators without a line through the bell, such as alert indicators 172B, indicate that the alert for the nearest virtual boundary are activated. Although not specifically illustrated, alert indicators 172 with a dashed line through the bell may indicate that the alert indicators are snoozed. The alert indicators 172 may also be selectable and/or manipulatable by the medical professional to activate or deactivate the alert assigned to the specific virtual boundaries, such as virtual boundaries (boundary 1, 2, 8, 9) and/or zone 1.

Referring to FIG. 11, the GUI 150F, the location of the virtual boundaries (boundary 1, 2) are shown in a revised pose, after user input to the planning buttons 168C to adjust the depth from 2 mm as shown in FIGS. 10 to 3 mm. Virtual boundaries (boundary 7,8) remain unchanged from FIGS. 10 to 11. Additionally, the medical professional has selected the alert button 156D configured to activate the various alerts, such as alerts 172B.

Once the medical professional is satisfied with the appearance of zone 1 and/or virtual boundaries (Boundary 1, 2, 8, 9), the medical professional may click the apply alert zones virtual button on the GUI 150F as shown in FIG. 11. By clicking the apply alert zones virtual button, the surgical navigation system 100 may propagate the various virtual alert zones as applied to the L3 vertebra to the remaining vertebra of the lumbar vertebra region 153.

Similar to as discussed with respect to the propagation of implant corrections, the navigation computer 140 may disable the navigation function discussed above with respect to the labels 174 when the medical professional selects the propagate button 197 of GUI 150F. The navigation computer 140 may be configured to when the medical professional selects the propagation button 197, prompt the medical professional to select one or more labels 174 associated with one or more desired vertebra from the lumbar vertebra region 153 to propagate the zone 1 to. In this manner, the navigation computer 140 is able to exclude a particular vertebra of the lumbar vertebra region 153 from propagation of implant corrections.

The medical professional may choose to forego propagation of the alert zone corrections to the remining vertebra by selecting the Approve L3 vertebra button 194 which will move onto alert zone planning for the remaining vertebra of the lumbar vertebra region 153. When the medical professional chooses the Approve L3 vertebra button, the default poses for the alert zones will correspond to the mapped poses for the alert zones based on the vertebra model 145.

The navigation computer 140 may include an alert zone correction database which stores alert zone and virtual boundary correction data (hereinafter, collectively referred to as alert zone correction data) and relevant patient data. The alert zone correction data may include spatial information, such as one or more transformations, which describe how the alert zones and/or virtual boundaries were corrected with respect to the initial suggested poses for the alert zone and/or virtual boundaries or spatial information which describes how the alert zones and/or virtual boundaries were corrected relative to the model alert zones and/or model virtual boundaries. The alert zone correction data may also include alert preferences and depth preferences for each alert zone.

The navigation computer 140 may access the alert zone corrections database to update a pose of the model alert zones and/or virtual boundaries of the vertebra model or to suggest alert zones and/or virtual boundaries for a future patient while taking into consideration the previous corrections for the medical professional. For example, the navigation computer 140 may be configured to determine one or more transformations between the initial mapped alert zones and/or virtual boundaries to L3 vertebra and the revised pose of the mapped alert zones and/or virtual boundaries of the L3 vertebra relative to the first coordinate system. In another example, the navigation computer 140, may store the depth adjustment made for each alert zone such as when the medical professional changes a depth setting (e.g., from 2 mm as shown in FIG. 10) to another depth setting (e.g., 3 mm depth as shown in FIG. 11). The navigation computer 140 may be configured to store the transformation data in the alert zone correction database which includes alert zone transformation data of other patients that the medical professional has previously operated on.

Based on the alert zone correction data stored in the alert zone correction database, the navigation computer 140 may periodically, such as when new patient cases entered into the alert zone corrections database reaches a threshold, retrieve the alert zone correction data for the patients from the alert zone correction database. The navigation computer 140 may, based on the retrieved alert zone correction data, determine an average alert zone transformation of all the alert zone transformation data stored in the alert zone correction database. For example, the navigation computer 140 would determine an average alert zone transformation for the alert zones (Zone 1-6). The navigation computer 140 may update the vertebra model 145 to reflect the average zone transformation. For example, when the number of patients reaches a threshold (e.g., one hundred patients) and the average transformation data indicates that the medical professional prefers a greater depth for zone 1 (i.e., a greater distance between virtual boundary 1 and virtual boundary 2), the navigation computer 140 may update the pose of model alert zone 1 of the vertebral model to reflect the preference of the medical professional for a greater depth.

The navigation computer 140 may in some instances may edit suggested alert zones and/or virtual boundaries based on the alert zone correction database. For example, after the navigation computer has mapped the model alert zones to the L3 vertebra as shown in FIG. 10, the medical professional may select the preference adjustment button 191 to update the L3 vertebra based on preferences from the medical professional determined based on the alert zone correction database.

The machine learning module that may be configured to train a machine learning model based on the alert zone correction database to provide suggested alert zones. For example, the machine learning module may train the machine learning model based on alert zone and/or virtual boundary corrections, alert settings and depth preferences, during previous surgical procedures and the outcomes for patients. The navigation computer 140 may be configured to process image data for each vertebra in the lumbar vertebra region 153 and other relevant patient data through the machine learning model to provide suggested alert zones based on historical preferences.

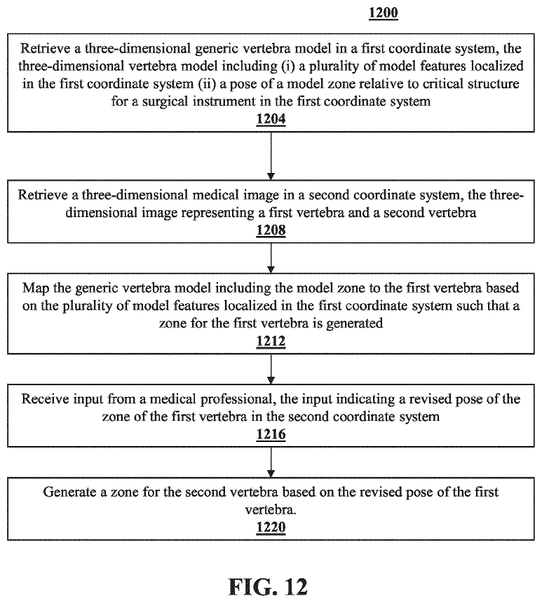

Referring to FIGS. 12-19, flowcharts illustrating methods according to the teachings of the present disclosure are described. As will be appreciated from the subsequent description below, the methods merely represent exemplary and non-limiting flowcharts that describe particular methods for implementing the teachings of the present disclosure. The methods may be implemented by the surgical navigation system 100 described above. The methods are in no way intended to serve as complete methods or catchall methods for implementing the various teachings discussed above.

With reference to FIG. 12, a method 1200 will be described. At 1204, where the method 1200 retrieves a three-dimensional vertebra model in a first coordinate system, the three-dimensional vertebra model including (i) a plurality of model features localized in the first coordinate system (ii) a pose of a model zone relative to critical structure for a surgical instrument in the first coordinate system. At 1208, the method 1200 retrieves a three-dimensional medical image in a second coordinate system, the three-dimensional image representing a first vertebra and a second vertebra. At 1212, the method 1200 maps the vertebra model including the model zone to the first vertebra based on the plurality of model features localized in the first coordinate system such that a zone for the first vertebra is generated. For example and without limitation, the vertebra model including the model zone may be mapped to a vertebra using the methodologies described in U.S. Patent Publication No. 2009/0089034 A1 and U.S. Provisional Patent Application No. 63/505,466, filed on Jun. 1, 2023 and published as PCT Patent Publication No. ______, the contents of each of which are hereby incorporated by reference herein in their entirety. At 1216, the method 1200 receives input from a medical professional, the input indicating a revised pose of the zone of the first vertebra in the second coordinate system. At 1220, the method 1200 generates a zone for the second vertebra based on the revised pose of the first vertebra.

In some implementations, the three-dimensional vertebra model may include poses of multiple model zones, with each model zone having a different pose in the coordinate system of the vertebra model. Assuming a three-dimensional image of multiple patient vertebrae, each model zone may be mapped to one of the patient vertebrae based on the plurality of model features localized in the coordinate system of the model such that a zone is generated for the one vertebra in the vertebra's coordinate system for each of the model zones, with each zone having a different pose. Then, responsive to receiving an input indicating a revised pose of any of the zones for the one vertebra in the coordinate system of the one vertebra, a zone may be generated for a different one of the patient vertebrae based on the revised pose of the zone for the previous vertebra.

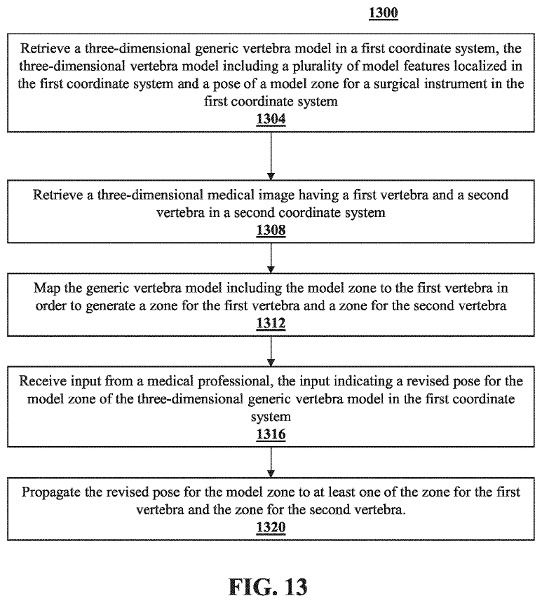

With reference to FIG. 13, a method 1300 will be described. At 1304, the method 1300 retrieves a three-dimensional vertebra model in a first coordinate system. The three-dimensional vertebra model including a plurality of model features localized in the first coordinate system and a pose of a model zone for a surgical instrument in the first coordinate system. At 1308, the method 1300 retrieves a three-dimensional medical image having a first vertebra and a second vertebra in a second coordinate system. At 1312, the method 1300 maps the vertebra model including the model zone to the first vertebra in order to generate a zone for the first vertebra and a zone for the second vertebra. At 1316, the method 1300 receive input from a medical professional, the input indicating a revised pose for the model zone of the three-dimensional vertebra model in the first coordinate system. At 1320, the method 1300 propagates the revised pose for the model zone to at least one of the zone for the first vertebra and the zone for the second vertebra.

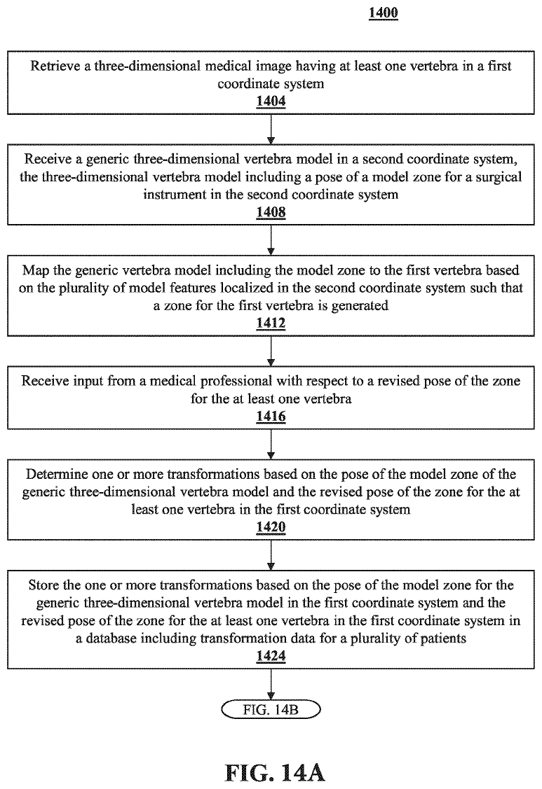

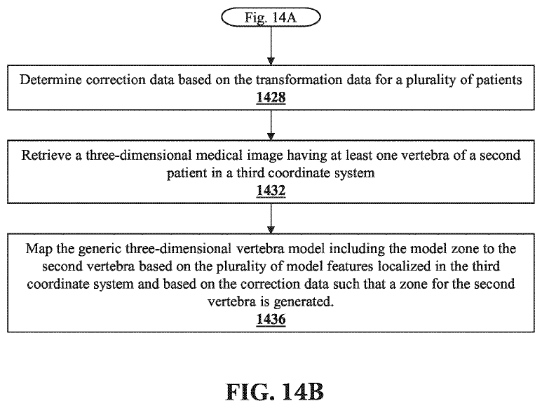

With reference to FIG. 14, a method 1400 will be described. At 1404, the method 1400 retrieves a three-dimensional medical image having at least one vertebra in a first coordinate system. At 1408, the method 900 receives a three-dimensional vertebra model in a second coordinate system. The three-dimensional vertebra model including a pose of a model zone for a surgical instrument in the second coordinate system. At 1412, the method 900 maps the vertebra model including the model zone to the first vertebra based on the plurality of model features localized in the second coordinate system such that a zone for the first vertebra is generated. At 1416, the method 900 receives input from a medical professional with respect to a revised pose of the zone for the at least one vertebra. At 1420, the method 900 determines one or more transformations based on the pose of the model zone of the three-dimensional vertebra model and the revised pose of the zone for the at least one vertebra in the first coordinate system. At 1424, the method 1400 store the one or more transformations based on the pose of the model zone for the three-dimensional vertebra model in the first coordinate system and the revised pose of the zone for the at least one vertebra in the first coordinate system in a database including transformation data for a plurality of patients. At 1428, the method 1400 determines correction data based on the transformation data for a plurality of patients. At 1432, the method 1400 retrieves a three-dimensional medical image having at least one vertebra of a second patient in a third coordinate system. At 1436, the method 1400 maps the three-dimensional vertebra model including the model zone to the second vertebra based on the plurality of model features localized in the third coordinate system and based on the correction data such that a zone for the second vertebra is generated.

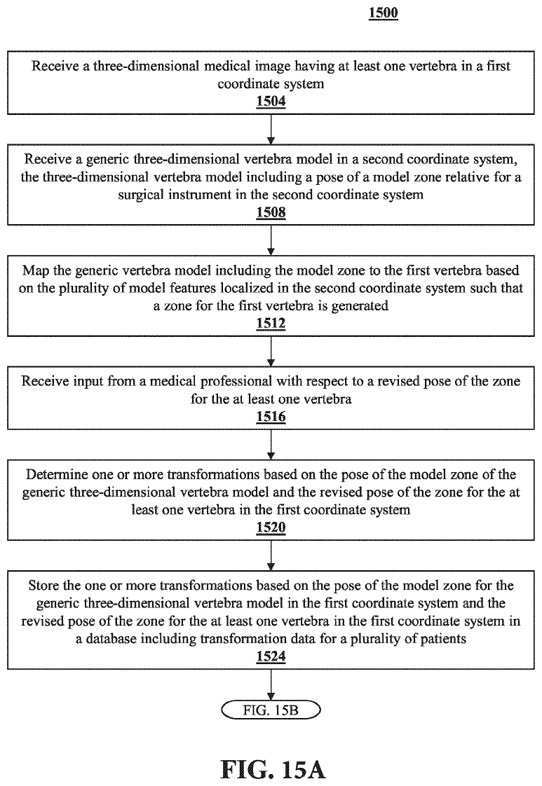

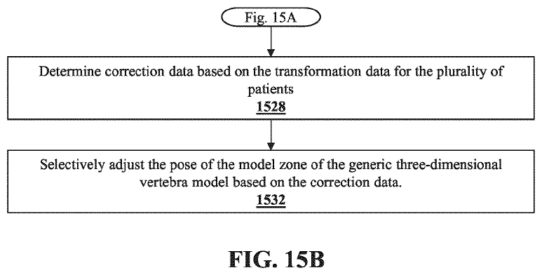

With reference to FIG. 15, a method 1500 will be described. At 1504, the method 1500 receives a three-dimensional medical image having at least one vertebra in a first coordinate system. At 1508, the method 1500 receives a three-dimensional vertebra model in a second coordinate system, the three-dimensional vertebra model including a pose of a model zone relative for a surgical instrument in the second coordinate system. At 1512, the method 1500 maps the vertebra model including the model zone to the first vertebra based on the plurality of model features localized in the second coordinate system such that a zone for the first vertebra is generated. At 1516, the method 1500 receives input from a medical professional with respect to a revised pose of the zone for the at least one vertebra. At 1520, the method 1500 determines one or more transformations based on the pose of the model zone of the three-dimensional vertebra model and the revised pose of the zone for the at least one vertebra in the first coordinate system. At 1524, the method 1500 store the one or more transformations based on the pose of the model zone for the three-dimensional vertebra model in the first coordinate system and the revised pose of the zone for the at least one vertebra in the first coordinate system in a database including transformation data for a plurality of patients. At 1528, the method 1500 determines correction data based on the transformation data for the plurality of patients. At 1532, the method 1500 selectively adjusts the pose of the model zone of the three-dimensional vertebra model based on the correction data, which may then be used to generate and/or adjust zones for patient vertebra going forward. For instance, responsive to receiving a further three-dimensional image of at least one vertebra in a third coordinate system, such as of another patient, the three-dimensional vertebra model including the adjusted pose of the model zone may be mapped to the at least one vertebra in the third coordinate system such that a zone is generated for the at least one vertebra of the further three-dimensional image.

In some instances, selectively adjusting the pose of the model zone of the three-dimensional vertebra model may be based on the number of the plurality of patients in which the correction data has been obtained. More specifically, this number may be compared to a predefine threshold, and the pose of the model zone of the three-dimensional vertebra model bay be adjusted based on the correction data is performed in response to the number of the plurality of patients exceeding the threshold.

In some instances, the three-dimensional vertebra model may include a pose of each of multiple model zones for monitoring a position of a surgical instrument during a surgical procedure. In general, given a three-dimensional image of at least one vertebra of a patient, the poses of the multiple model zones of the three-dimensional vertebra model may be defined such that that pose of each model zone mapped to the at least one vertebra generates a zone with different coordinates in the coordinate system of the at least one vertebra of the patient. Moreover, the pose of each model zone of the three-dimensional vertebra model may be individually selectively adjusted responsive to input being received that indicates a revised pose of the zone for the at least one vertebra of the patient that corresponds to the model zone. In other words, responsive to input indicating a revised pose of the zone generated for the at least one patient vertebra that corresponds to any one of the model zones, one or more transformations based on the pose of that model zone in the coordinate system of the three-dimensional vertebra model and the revised pose of the zone in the coordinate system of the three-dimensional image of the patient may be determined and stored in a database as described above. Correction data based on the transformation data for a plurality of patients that corresponds to that specific model zone may then be determined and used to selective adjust the pose of that model zone of the three-dimensional vertebra model.

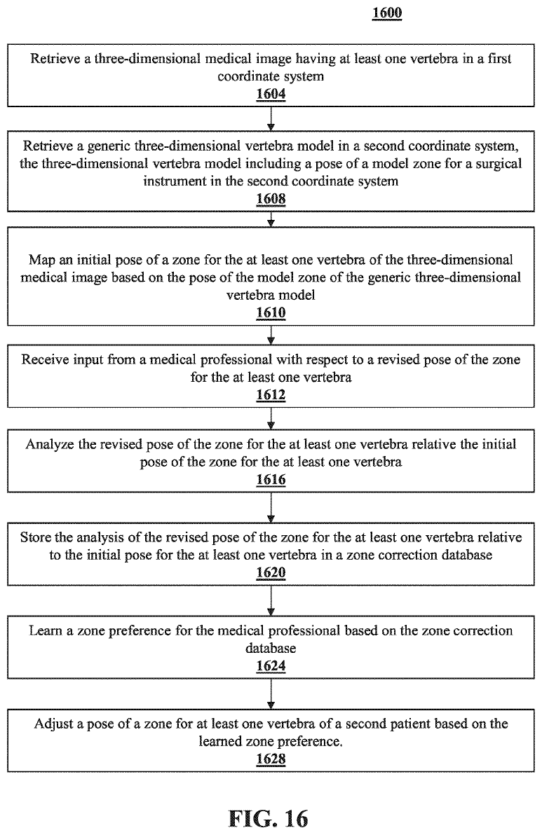

With reference to FIG. 16, a method 1600 will be described. At 1604, the method 1600 retrieves a three-dimensional medical image having at least one vertebra in a first coordinate system. At 1608, the method 1600 retrieves a three-dimensional vertebra model in a second coordinate system, the three-dimensional vertebra model including a pose of a model zone for a surgical instrument in the second coordinate system. At 1610, the method 1600 maps an initial pose of a zone for the at least one vertebra of the three-dimensional medical image based on the pose of the model zone of the three-dimensional vertebra model. At 1612, the method 1600 receives input from a medical professional with respect to a revised pose of the zone for the at least one vertebra. At 1616, the method 1600 analyzes the revised pose of the zone for the at least one vertebra relative the initial pose of the zone for the at least one vertebra. At 1620, the method 1600 stores the analysis of the revised pose of the zone for the at least one vertebra relative to the initial pose for the at least one vertebra in a zone correction database. At 1624, the method 1600 learns a zone preference for the medical professional based on the zone correction database.

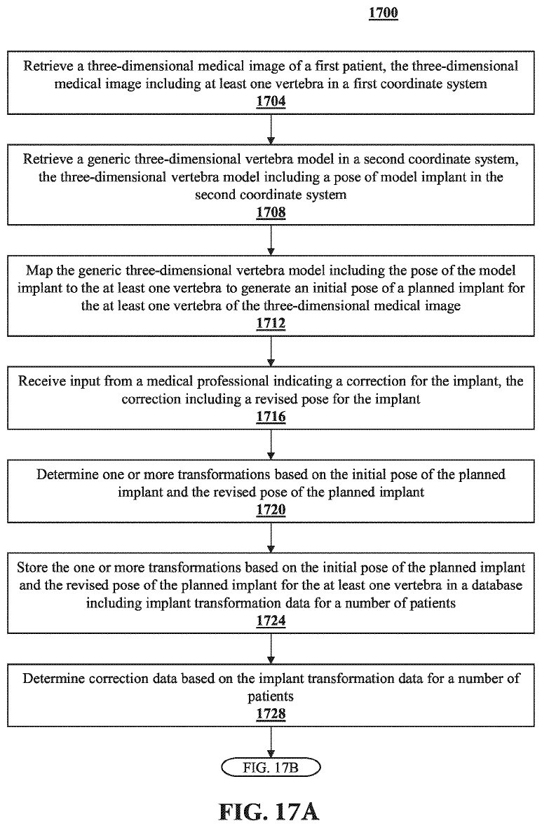

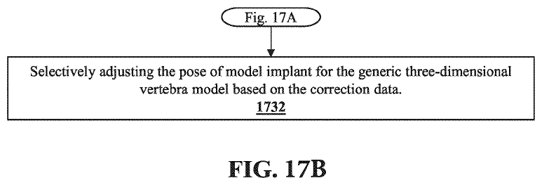

With reference to FIG. 17, a method 1700 will be described. At 1704, the method 1700 retrieve a three-dimensional medical image of a first patient, the three-dimensional medical image including at least one vertebra in a first coordinate system. At 1708, the method 1700 retrieves a three-dimensional vertebra model in a second coordinate system, the three-dimensional vertebra model including a pose of model implant in the second coordinate system. At 1712, the method 1700, maps the three-dimensional vertebra model including the pose of the model implant to the at least one vertebra to generate an initial pose of a planned implant for the at least one vertebra of the three-dimensional medical image. At 1716, the method 1700 receives input from a medical professional indicating a correction for the implant, the correction including a revised pose for the implant. At 1720, the method 1700 determines one or more transformations based on the initial pose of the planned implant and the revised pose of the planned implant. At 1724, the method 1700 stores the one or more transformations based on the initial pose of the planned implant and the revised pose of the planned implant for the at least one vertebra in a database including implant transformation data for a number of patients. At 1728, the method 1700 determines correction data based on the implant transformation data for a number of patients. At 1732, the method 1700 selectively adjusts the pose of model implant for the three-dimensional vertebra model based on the correction data.

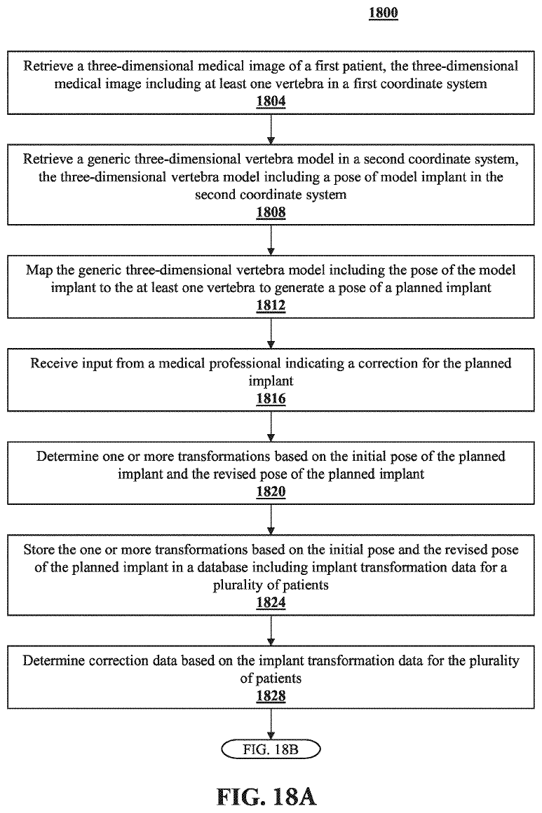

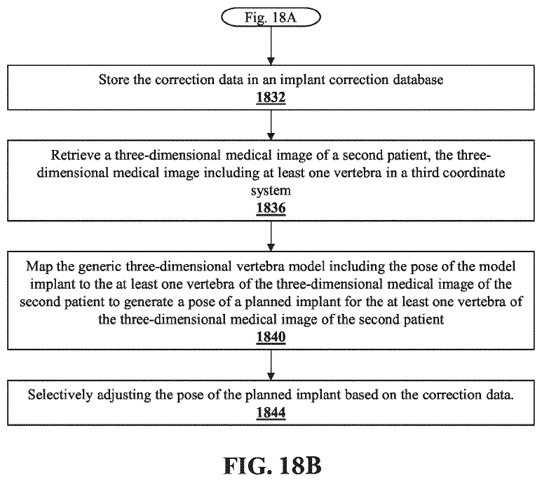

With reference to FIG. 18, a method 1800 will be described. At 1804, the method 1800 retrieves a three-dimensional medical image of a first patient, the three-dimensional medical image including at least one vertebra in a first coordinate system. At 1808, the method 1800 retrieves a three-dimensional vertebra model in a second coordinate system, the three-dimensional vertebra model including a pose of model implant in the second coordinate system. At 1812, the method 1800 maps the three-dimensional vertebra model including the pose of the model implant to the at least one vertebra to generate a pose of a planned implant. At 1816, the method 1800 receives input from a medical professional indicating a correction for the planned implant. At 1820, the method 1800 determines one or more transformations based on the initial pose of the planned implant and the revised pose of the planned implant. At 1824, the method 1800 stores the one or more transformations based on the initial pose and the revised pose of the planned implant in a database including implant transformation data for a plurality of patients. At 1828, the method 1800 determine correction data based on the implant transformation data for the plurality of patients. At 1832, the method 1800 stores the correction data in an implant correction database. At 1836, the method 1800 retrieves a three-dimensional medical image of a second patient, the three-dimensional medical image including at least one vertebra in a third coordinate system. At 1840, the method 1800 maps the three-dimensional vertebra model including the pose of the model implant to the at least one vertebra of the three-dimensional medical image of the second patient to generate a pose of a planned implant for the at least one vertebra of the three-dimensional medical image of the second patient. At 1844, the method 1800 selectively adjusts the pose of the planned implant based on the correction data.

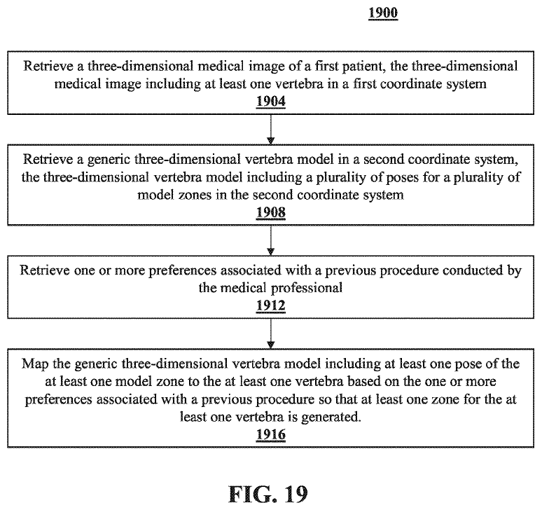

With reference to FIG. 19, a method 1900 will be described. At 1904, the method 1900 retrieves a three-dimensional medical image of a first patient, the three-dimensional medical image including at least one vertebra in a first coordinate system. At 1908, the method 1900 retrieves a three-dimensional vertebra model in a second coordinate system, the three-dimensional vertebra model including a plurality of poses for a plurality of model zones in the second coordinate system. At 1912, the method 1900 retrieves one or more preferences associated with a previous procedure conducted by the medical professional. For example and without limitation, the one or more preferences associated with a previous procedure conducted by the medical professional may include a selection of one or more model zones form the plurality of model zones during the previous procedure, and/or a margin previously selected by the medical professional to define a zone used in the previous procedure. At 1916, the method 1900 maps the three-dimensional vertebra model including at least one pose of the at least one model zone to the at least one vertebra based on the one or more preferences associated with a previous procedure so that at least one zone for the at least one vertebra is generated.

### Clauses

Clause 1-A method for mapping zones for controlling a surgical instrument to a three-dimensional medical image, the method comprising: retrieving a three-dimensional bone model in a first coordinate system, the three-dimensional bone model including (i) a plurality of model features localized in the first coordinate system and (ii) a pose of a model zone relative to critical structure for a surgical instrument in the first coordinate system; retrieving a three-dimensional medical image in a second coordinate system, the three-dimensional image representing a first bone region and a second bone region; mapping the bone model including the model zone to the first bone region based on the plurality of model features localized in the first coordinate system such that a zone for the first bone region is generated; receiving input from a medical professional, the input indicating a revised pose of the zone of the first bone region in the second coordinate system; and generating a zone for the second bone region based on the revised pose of the zone of the first bone region.

Clause 2-A method for mapping zones for a surgical instrument to a three-dimensional medical image, the method comprising: retrieving a three-dimensional bone model in a first coordinate system, the three-dimensional bone model including a plurality of model features localized in the first coordinate system and a pose of a model zone for a surgical instrument in the first coordinate system; retrieving a three-dimensional medical image having a first bone region and a second bone region in a second coordinate system; mapping the bone model including the model zone to the first bone region in order to generate a zone for the first bone region and a zone for the second bone region; receiving input from a medical professional, the input indicating a revised pose for the model zone of the three-dimensional bone model in the first coordinate system; and propagating the revised pose for the model zone to at least one of the zone for the first bone region and the zone for the second bone region.

Clause 3-A method for mapping zones for a surgical instrument to a three-dimensional medical image, the method comprising: retrieving a three-dimensional bone model in a first coordinate system, the three-dimensional bone model including (i) a plurality of model features localized in the first coordinate system and (ii) a model zone for a surgical instrument in the first coordinate system; retrieving a three-dimensional medical image of a first patient having at least one bone in a second coordinate system; mapping the three-dimensional bone model including the model zone to the at least one bone to generate a zone relative the at least one bone; receiving input with respect to a revised pose of the zone for the at least one bone; revising the pose of the model zone based on the revised pose of the zone for the at least one bone; retrieving a three-dimensional medical image of a second patient having at least one bone in a third coordinate system; and mapping the three-dimensional vertebra model including the revised pose of the model zone to the at least one bone of the second patient in order to generate a zone for the at least one bone of the second patient.

Clause 4-A method for adjusting a zone for a three-dimensional medical image according to historical preference of a medical professional, the method comprising: retrieving a three-dimensional medical image having at least one bone in a first coordinate system; receiving a three-dimensional bone model in a second coordinate system, the three-dimensional bone model including a pose of a model zone for a surgical instrument in the second coordinate system; mapping the bone model including the model zone to the first bone based on the plurality of model features localized in the second coordinate system such that a zone for the first bone is generated; receiving input from a medical professional with respect to a revised pose of the zone for the at least one bone; determining one or more transformations based on the pose of the model zone of the three-dimensional bone model and the revised pose of the zone for the at least one bone in the first coordinate system; storing the one or more transformations based on the pose of the model zone for the three-dimensional bone model in the first coordinate system and the revised pose of the zone for the at least one bone in the first coordinate system in a database including transformation data for a plurality of patients; determining correction data based on the transformation data for a plurality of patients; retrieving a three-dimensional medical image having at least one bone of a second patient in a third coordinate system; and mapping the three-dimensional bone model including the model zone to the second bone based on the plurality of model features localized in the third coordinate system and based on the correction data such that a zone for the second bone is generated.

Clause 5-A method for adjusting a zone for a three-dimensional medical image according to historical preference of a medical professional, the method comprising: receiving a three-dimensional medical image having at least one bone in a first coordinate system; receiving a three-dimensional bone model in a second coordinate system, the three-dimensional bone model including a pose of a model zone relative for a surgical instrument in the second coordinate system; mapping the bone model including the model zone to a first bone region based on the plurality of model features localized in the second coordinate system such that a zone for the first bone region is generated; receiving input from a medical professional with respect to a revised pose of the zone for the at least one bone; determining one or more transformations based on the pose of the model zone of the three-dimensional bone model and the revised pose of the zone for the at least one bone in the first coordinate system; storing the one or more transformations based on the pose of the model zone for the three-dimensional bone model in the first coordinate system and the revised pose of the zone for the at least one bone in the first coordinate system in a database including transformation data for a plurality of patients; determining correction data based on the transformation data for the plurality of patients; and selectively adjusting the pose of the model zone of the three-dimensional bone model based on the correction data.

Clause 6-A method for adjusting a zone for a three-dimensional medical image according to historical preference of a medical professional, the method comprising: retrieving a three-dimensional medical image having at least one bone in a first coordinate system; retrieving a three-dimensional bone model in a second coordinate system, the three-dimensional bone model including a pose of a model zone for a surgical instrument in the second coordinate system, mapping an initial pose of a zone for the at least one bone of the three-dimensional medical image based on the pose of the model zone of the three-dimensional bone model; receiving input from a medical professional with respect to a revised pose of the zone for the at least one bone; analyzing the revised pose of the zone for the at least one bone relative the initial pose of the zone for the at least one bone; storing the analysis of the revised pose of the zone for the at least one bone relative to the initial pose for the at least one bone in a zone correction database; learning a zone preference for the medical professional based on the zone correction database; and adjusting a pose of a zone for at least one bone of a second patient based on the learned zone preference.

Clause 7-A method for adjusting a planned implant based on historical preference of a medical professional, the method comprising: retrieving a three-dimensional medical image of a first patient, the three-dimensional medical image including at least one bone in a first coordinate system; retrieving a three-dimensional bone model in a second coordinate system, the three-dimensional bone model including a pose of model implant in the second coordinate system; mapping the three-dimensional bone model including the pose of the model implant to the at least one bone to generate an initial pose of a planned implant for the at least one bone of the three-dimensional medical image; receiving input from a medical professional indicating a correction for the implant, the correction including a revised pose for the implant; determining one or more transformations based on the initial pose of the planned implant and the revised pose of the planned implant; storing the one or more transformations based on the initial pose of the planned implant and the revised pose of the planned implant for the at least one bone in a database including implant transformation data for a number of patients; determining correction data based on the implant transformation data for a number of patients; and selectively adjusting the pose of model implant for the three-dimensional bone model based on the correction data.

Clause 8-A method for adjusting a planned implant pose based on historical preference of a medical professional, the method comprising: retrieving a three-dimensional medical image of a first patient, the three-dimensional medical image including at least one bone in a first coordinate system; retrieving a three-dimensional bone model in a second coordinate system, the three-dimensional bone model including a pose of model implant in the second coordinate system; mapping the three-dimensional bone model including the pose of the model implant to the at least one bone to generate a pose of a planned implant; receiving input from a medical professional indicating a correction for the planned implant; determining one or more transformations based on the initial pose of the planned implant and the revised pose of the planned implant; storing the one or more transformations based on the initial pose and the revised pose of the planned implant in a database including implant transformation data for a plurality of patients; determining correction data based on the implant transformation data for the plurality of patients; storing the correction data in an implant correction database; retrieving a three-dimensional medical image of a second patient, the three-dimensional medical image including at least one bone in a third coordinate system; mapping the three-dimensional bone model including the pose of the model implant to the at least one bone of the three-dimensional medical image of the second patient to generate a pose of a planned implant for the at least one bone of the three-dimensional medical image of the second patient; and selectively adjusting the pose of the planned implant based on the correction data.

Clause 9-A method for adjusting a zone based on historical preference of a medical professional, the method comprising: retrieving a three-dimensional medical image of a first patient, the three-dimensional medical image including at least one bone in a first coordinate system; retrieving a three-dimensional bone model in a second coordinate system, the three-dimensional bone model including a plurality of poses for a plurality of model zones in the second coordinate system; retrieving one or more preferences associated with a previous procedure conducted by the medical professional; and mapping the three-dimensional vertebra model including at least one pose of the at least one model zone to the at least one bone based on the one or more preferences associated with a previous procedure so that at least one zone for the at least one bone is generated.

The foregoing description is merely illustrative in nature and is in no way intended to limit the disclosure, its application, or uses. The broad teachings of the disclosure can be implemented in a variety of forms. Therefore, while this disclosure includes particular examples, the true scope of the disclosure should not be so limited since other modifications will become apparent upon a study of the drawings, the specification, and the following claims. It should be understood that one or more steps within a method may be executed in different order (or concurrently) without altering the principles of the present disclosure. Further, although each of the examples is described above as having certain features, any one or more of those features described with respect to any example of the disclosure can be implemented in and/or combined with features of any of the other examples, even if that combination is not explicitly described. In other words, the described examples are not mutually exclusive, and permutations of one or more examples with one another remain within the scope of this disclosure.

Spatial and functional relationships between elements (for example, between controllers, circuit elements, semiconductor layers, etc.) are described using various terms, including “connected,” “engaged,” “coupled,” “adjacent,” “next to,” “on top of,” “above,” “below,” and “disposed.” Unless explicitly described as being “direct,” when a relationship between first and second elements is described in the above disclosure, that relationship can be a direct relationship where no other intervening elements are present between the first and second elements, but can also be an indirect relationship where one or more intervening elements are present (either spatially or functionally) between the first and second elements.

As used herein, the phrase at least one of A, B, and C should be construed to mean a logical (A OR B OR C), using a non-exclusive logical OR, and should not be construed to mean “at least one of A, at least one of B, and at least one of C.” The term subset does not necessarily require a proper subset. In other words, a first subset of a first set may be coextensive with (equal to) the first set.

In the FIGS., the direction of an arrow, as indicated by the arrowhead, generally demonstrates the flow of information (such as data or instructions) that is of interest to the illustration. For example, when element A and element B exchange a variety of information, but information transmitted from element A to element B is relevant to the illustration, the arrow may point from element A to element B. This unidirectional arrow does not imply that no other information is transmitted from element B to element A. Further, for information sent from element A to element B, element B may send requests for, or receipt acknowledgements of, the information to element A.

In this application, including the definitions below, the term “controller” or “module” may be replaced with the term “circuit.” The term “controller” may refer to, be part of, or include: an Application Specific Integrated Circuit (ASIC); a programmable system on a chip (PSoC); a digital, analog, or mixed analog/digital discrete circuit; a digital, analog, or mixed analog/digital integrated circuit; a combinational logic circuit; a field programmable gate array (FPGA); a processor circuit (shared, dedicated, or group) that executes code; a memory circuit (shared, dedicated, or group) that stores code executed by the processor circuit; other suitable hardware components that provide the described functionality; or a combination of some or all of the above, such as in a system-on-chip.

The controller may include one or more interface circuits with one or more transceivers. In some examples, the interface circuit(s) may implement wired or wireless interfaces that connect to a local area network (LAN) or a wireless personal area network (WPAN). Examples of a LAN are Institute of Electrical and Electronics Engineers (IEEE) Standard 802.11-2016 (also known as the WIFI wireless networking standard) and IEEE Standard 802.3-2015 (also known as the ETHERNET wired networking standard). Examples of a WPAN are the BLUETOOTH wireless networking standard from the Bluetooth Special Interest Group and IEEE Standard 802.15.4.

The controller may communicate with other controllers using the interface circuit(s). Although the controller may be depicted in the present disclosure as logically communicating directly with other controllers, in various implementations the controller may actually communicate via a communications system. The communications system may include physical and/or virtual networking equipment such as hubs, switches, routers, gateways and transceivers. In some implementations, the communications system connects to or traverses a wide area network (WAN) such as the Internet. For example, the communications system may include multiple LANs connected to each other over the Internet or point-to-point leased lines using technologies including Multiprotocol Label Switching (MPLS) and virtual private networks (VPNs).

In various implementations, the functionality of the controller may be distributed among multiple controllers that are connected via the communications system. For example, multiple controllers may implement the same functionality distributed by a load balancing system. In a further example, the functionality of the controller may be split between a server (also known as remote, or cloud) controller and a client (or, user) controller.

Some or all hardware features of a controller may be defined using a language for hardware description, such as IEEE Standard 1364-2005 (commonly called “Verilog”) and IEEE Standard 1076-2008 (commonly called “VHDL”). The hardware description language may be used to manufacture and/or program a hardware circuit. In some implementations, some or all features of a controller may be defined by a language, such as IEEE 1666-2005 (commonly called “SystemC”), that encompasses both code, as described below, and hardware description.

The term code, as used above, may include software, firmware, and/or microcode, and may refer to programs, routines, functions, classes, data structures, and/or objects. The term shared processor circuit encompasses a single processor circuit that executes some or all code from multiple controllers. The term group processor circuit encompasses a processor circuit that, in combination with additional processor circuits, executes some or all code from one or more controllers. References to multiple processor circuits encompass multiple processor circuits on discrete dies, multiple processor circuits on a single die, multiple cores of a single processor circuit, multiple threads of a single processor circuit, or a combination of the above. The term shared memory circuit encompasses a single memory circuit that stores some or all code from multiple controllers. The term group memory circuit encompasses a memory circuit that, in combination with additional memories, stores some or all code from one or more controllers.

The term memory circuit is a subset of the term computer-readable medium. The term computer-readable medium, as used herein, does not encompass transitory electrical or electromagnetic signals propagating through a medium (such as on a carrier wave); the term computer-readable medium may therefore be considered tangible and non-transitory. Non-limiting examples of a non-transitory computer-readable medium are nonvolatile memory circuits (such as a flash memory circuit, an erasable programmable read-only memory circuit, or a mask read-only memory circuit), volatile memory circuits (such as a static random access memory circuit or a dynamic random access memory circuit), magnetic storage media (such as an analog or digital magnetic tape or a hard disk drive), and optical storage media (such as a CD, a DVD, or a Blu-ray Disc).

The apparatuses and methods described in this application may be partially or fully implemented by a special purpose computer created by configuring a general purpose computer to execute one or more particular functions embodied in computer programs. The functional blocks and flowchart elements described above may serve as software specifications, which can be translated into the computer programs by the routine work of a skilled technician or programmer.

The computer programs include processor-executable instructions that are stored on at least one non-transitory computer-readable medium. The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc.

The computer programs may include: (i) descriptive text to be parsed, such as HTML (hypertext markup language), XML (extensible markup language), or JSON (JavaScript Object Notation), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C #, Objective-C, Swift, Haskell, Go, SQL, R, Lisp, Java®, Fortran, Perl, Pascal, Curl, OCaml, JavaScript®, HTML5 (Hypertext Markup Language 5th revision), Ada, ASP (Active Server Pages), PHP (PHP: Hypertext Preprocessor), Scala, Eiffel, Smalltalk, Erlang, Ruby, Flash®, Visual Basic®, Lua, MATLAB, SIMULINK, and Python®.

1. A method for mapping zones for monitoring a position of a surgical instrument during a procedure to a three-dimensional medical image of patient vertebrae, the method comprising:
receiving a three-dimensional vertebra model in a first coordinate system, the three-dimensional vertebra model including a plurality of model features localized in the first coordinate system and a pose of a model zone for the surgical instrument in the first coordinate system;
receiving a three-dimensional image in a second coordinate system, the three-dimensional image representing a first vertebra and a second vertebra of a patient;
mapping the three-dimensional vertebra model including the model zone to the first vertebra based on the plurality of model features localized in the first coordinate system such that a zone for the first vertebra is generated in the second coordinate system;
receiving input from a medical professional, the input indicating a revised pose of the zone for the first vertebra in the second coordinate system; and
generating a zone for the second vertebra based on the revised pose of the zone for the first vertebra.

2. The method of claim 1, wherein generating the zone for the second vertebra based on the revised pose of the zone for the first vertebra includes:
mapping the three-dimensional vertebra model including the model zone to the second vertebra based on the plurality of model features localized in the first coordinate system such that the zone for the second vertebra is generated in the second coordinate system; and
editing the zone for the second vertebra based on the revised pose of the zone for the first vertebra.

3. The method of claim 1, wherein the zone for the first vertebra and the zone for the second vertebra are defined by a first boundary relative to a critical structure and a second boundary spaced a first distance from the first boundary, the first boundary and the second boundary defining a volume representing the zone.

4. The method of claim 1, wherein the zone for the first vertebra is provided as a user-selectable object, the input corresponding to a manipulation of the user-selectable object.

5. The method of claim 4, wherein mapping the three-dimensional vertebra model including the model zone to the first vertebra based on the plurality of model features localized in the first coordinate system such that the zone for the first vertebra is generated in the second coordinate system includes segmenting the first vertebra within the three-dimensional image.

6. The method of claim 1, comprising:
tracking a pose of the surgical instrument; and
performing at least one of controlling a parameter of the surgical instrument and providing an alert based on the tracked pose of the surgical instrument relative to the zone for the first vertebra and the zone for the second vertebra.

7. The method of claim 1, wherein the zone for the first vertebra and the zone for the second vertebra are provided as meshes.

8. The method of claim 1, wherein the three-dimensional medical image represents a third vertebra and a fourth vertebra, the method comprising:
receiving input with respect to at least one of the third vertebra and the fourth vertebra; and
generating a zone for the third vertebra or a zone for the fourth vertebra in response to the input with respect to the at least one of the third vertebra and the fourth vertebra and based on the revised pose of the zone for the first vertebra.

9. The method of claim 1, wherein the model zone is defined as a first model zone, the zone for the first vertebra is defined as a first zone for the first vertebra, the three-dimensional vertebra model in the first coordinate system includes a pose of a second model zone for the surgical instrument in the first coordinate system, and the second model zone has a different pose than the first model zone in the first coordinate system, the method comprising:
mapping the second model zone to the first vertebra based on the plurality of model features localized in the first coordinate system such that a second zone for the first vertebra is generated in the second coordinate system, the second zone for the first vertebra having a different pose than the first zone for the first vertebra;
receiving input from the medical professional, the input indicating a revised pose of the second zone for the first vertebra in the second coordinate system; and
generating a second zone for the second vertebra based on the revised pose of the second zone for the first vertebra.

10. The method of claim 1, comprising updating the pose of the model zone in the first coordinate system based on the revised pose of the zone for the first vertebra.

11-30. (canceled)

31. A surgical navigation system for monitoring a position of a surgical instrument during a procedure, the surgical navigation system comprising:
a tracking unit;
a user input device;
an alert device; and
at least one controller in communication with the tracking unit, the user input device, and the alert device, the at least one controller configured to:
receive a three-dimensional vertebra model in a first coordinate system, the three-dimensional vertebra model including a plurality of model features localized in the first coordinate system and a pose of a model zone for the surgical instrument in the first coordinate system;
receive a three-dimensional image in a second coordinate system, the three-dimensional image representing a first vertebra and a second vertebra of a patient;
map the three-dimensional vertebra model including the model zone to the first vertebra based on the plurality of model features localized in the first coordinate system such that a zone for the first vertebra is generated in the second coordinate system;
receive input from the user input device, the input indicating a revised pose of the zone for the first vertebra in the second coordinate system;
generate a zone for the second vertebra based on the revised pose of the zone for the first vertebra;
receive a pose of the surgical instrument from the tracking unit; and
perform at least one of controlling a parameter of the surgical instrument and causing the alert device to provide an alert based on the tracked pose of the surgical instrument relative to the zone for the first vertebra and the zone for the second vertebra.

32. The surgical navigation system of claim 31, wherein the at least one controller is configured to generate the zone for the second vertebra based on the revised pose of the zone for the first vertebra by:
mapping the three-dimensional vertebra model including the model zone to the second vertebra based on the plurality of model features localized in the first coordinate system such that the zone for the second vertebra is generated in the second coordinate system; and
editing the zone for the second vertebra based on the revised pose of the zone for the first vertebra.

33. The surgical navigation system of claim 31, wherein the zone for the first vertebra and the zone for the second vertebra are defined by a first boundary relative to a critical structure and a second boundary spaced a first distance from the first boundary, the first boundary and the second boundary defining a volume representing the zone.

34. The surgical navigation system of claim 31, wherein:
the zone for the first vertebra is provided as a user-selectable object; and
the input corresponding to a manipulation of the user-selectable object.

35. The surgical navigation system of claim 31, wherein the at least one controller is configured to:
perform a segmentation of the first vertebra within the three-dimensional image; and
map the three-dimensional vertebra model including the model zone to the first vertebra based on the plurality of model features localized in the first coordinate system such that the zone for the first vertebra is generated in the second coordinate system based on the segmentation of the first vertebra within the three-dimensional image.

36. The surgical navigation system of claim 31, wherein:
the three-dimensional image represents a third vertebra and a fourth vertebra; and
the at least one controller is configured to:
receive input with respect to at least one of the third vertebra and the fourth vertebra from the user input device; and
generate a zone for the third vertebra or a zone for the fourth vertebra in response to the input with respect to the at least one of the third vertebra and the fourth vertebra and based on the revised pose of the zone for the first vertebra.

37. The surgical navigation system of claim 31, wherein:
the model zone is defined as a first model zone;
the zone for the first vertebra is defined as a first zone for the first vertebra;
the three-dimensional vertebra model in the first coordinate system includes a pose of a second model zone for the surgical instrument in the first coordinate system;
the second model zone has a different pose than the first model zone in the first coordinate system; and
the at least one controller is configured to:
map the second model zone to the first vertebra based on the plurality of model features localized in the first coordinate system such that a second zone for the first vertebra is generated in the second coordinate system, the second zone for the first vertebra having a different pose than the first zone for the first vertebra;
receive input from user input device, the input indicating a revised pose of the second zone for the first vertebra in the second coordinate system; and
generate a second zone for the second vertebra based on the revised pose of the second zone for the first vertebra.

38. The surgical navigation system of claim 31, wherein the at least one controller is configured to update the pose of the model zone in the first coordinate system based on the revised pose of the zone for the first vertebra.

39. The surgical navigation system of claim 31, further comprising a display unit configured to display a graphical user interface;
wherein the input to the user input device includes an interaction with the graphical user interface.

40. A method for mapping zones for monitoring a position of a surgical instrument during a procedure to a three-dimensional image of at least one patient vertebra, the method comprising:
receiving a three-dimensional vertebra model in a first coordinate system, the three-dimensional vertebra model including a plurality of model features localized in the first coordinate system and a pose of a model zone for the surgical instrument in the first coordinate system;
receiving a three-dimensional image of at least one vertebra of a first patient in a second coordinate system;
mapping the three-dimensional vertebra model including the model zone to the at least one vertebra based on the plurality of model features localized in the first coordinate system to generate a zone for the at least one vertebra in the second coordinate system;
receiving input with respect to a revised pose of the zone for the at least one vertebra in the second coordinate system;
revising the pose of the model zone in the first coordinate system based on the revised pose of the zone for the at least one vertebra;
receiving a three-dimensional image of at least one vertebra of a second patient in a third coordinate system; and
mapping the three-dimensional vertebra model including the revised pose of the model zone to the at least one vertebra of the second patient based on the plurality of model features localized in the first coordinate system to generate a zone for the at least one vertebra of the second patient in the third coordinate system.
